(19) United States
(12) Patent Application Publication
(10) Pub. No.: US 2025/0034596 A1
(43) Pub. Date: Jan. 30, 2025

(54) Omni-103 CRISPR Nuclease-RNA Complexes

(71) Applicant: EmendoBio Inc., Wimington, DE (US)

(72) Inventors: Lior IZHAR, Tel Aviv (IL); Liat ROCKAH, Rishon LeZion (IL); Nadav MARBACH BAR, Rehovot (IL); Nir HECHT, Tel Aviv (IL)

(21) Appl. No.: 18/717,266

(22) PCT Filed: Dec. 6, 2022
(86) PCT No.: PCT/US2022/081019

§ 371 Date: Jun. 6, 2024

Related U.S. Application Data
(60) Provisional application No. 63/286,855, filed on Dec. 7, 2021.

(51) Int. Cl. C12N 15/90; C12N 9/22; C12N 15/11

(52) U.S. Cl. CPC C12N 15/907; C12N 9/22; C12N 15/11; C12N 2310/20; C12N 2310/531

(57) ABSTRACT

A composition comprising a non-naturally occurring RNA molecule, the RNA molecule comprising an RNA scaffold portion, the RNA scaffold portion having the structure: crRNA repeat sequence portion-tracrRNA portion: wherein the RNA scaffold portion forms a complex with and targets an OMNI-103 CRISPR nuclease to a DNA target site having complementarity to a guide sequence portion of the RNA molecule.

This application claims the benefit of U.S. Provisional Application No. 63/286,855, filed Dec. 7, 2021, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “221206_91822-A-PCT_Sequence_Listing_AWG.xml”, which is 81 kilobytes in size, and which was created on Nov. 9, 2022 in the IBM-PC machine format, having an operating system compatibility with MS-Windows, which is contained in the XML file filed Dec. 6, 2022 as part of this application.

### FIELD OF THE INVENTION

The present invention is directed to, inter alia, composition and methods for genome editing.

### BACKGROUND OF THE INVENTION

The Clustered Regularly Interspaced Short Palindromic Repeat (CRISPR) systems of bacterial and archaeal adaptive immunity show extreme diversity of protein composition and genomic loci architecture. The CRISPR systems have become important tools for research and genome engineering. Nevertheless, many details of CRISPR systems have not been determined and the applicability of CRISPR nucleases may be limited by sequence specificity requirements, expression, or delivery challenges. Different CRISPR nucleases have diverse characteristics such as: size, PAM site, on target activity, specificity, cleavage pattern (e.g. blunt, staggered ends), and prominent pattern of indel formation following cleavage. Different sets of characteristics may be useful for different applications. For example, some CRISPR nucleases may be able to target particular genomic loci that other CRISPR nucleases cannot due to limitations of the PAM site. In addition, some CRISPR nucleases currently in use exhibit pre-immunity, which may limit in vivo applicability. See Charlesworth et al., Nature Medicine (2019) and Wagner et al., Nature Medicine (2019). Accordingly, discovery, engineering, and improvement of novel CRISPR nucleases and the RNA molecules that activate and target them is of importance.

### SUMMARY OF THE INVENTION

The invention provides a composition comprising a non-naturally occurring RNA molecule, the RNA molecule comprising a crRNA repeat sequence portion and a guide sequence portion, wherein the RNA molecule forms a complex with and targets an OMNI-103 nuclease to a DNA target site in the presence of a tracrRNA sequence, wherein the tracrRNA sequence is encoded by a tracrRNA portion of the RNA molecule or a tracrRNA portion of a second RNA molecule.

The invention also provides a composition comprising a non-naturally occurring RNA molecule, the RNA molecule comprising an RNA scaffold portion, the RNA scaffold portion having the structure:
crRNA repeat sequence portion-tracrRNA portion;
   wherein the RNA scaffold portion forms a complex with and targets an OMNI-103 CRISPR nuclease to a DNA target site having complementarity to a guide sequence portion of the RNA molecule.

Disclosed herein are compositions and methods that may be utilized for genomic engineering, epigenomic engineering, genome targeting, genome editing of cells, and/or in vitro diagnostics using an OMNI-103 CRISPR nuclease and a non-naturally occurring RNA molecule comprising a scaffold portion capable of specifically binding and activating the OMNI-103 CRISPR nuclease to target a DNA target site based on a guide sequence portion, also referred to as a RNA spacer portion, of the RNA molecule.

The disclosed compositions may be utilized for modifying genomic DNA sequences. As used herein, genomic DNA refers to linear and/or chromosomal DNA and/or plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-strand breaks (DSBs) at pre-determined target sites in a genomic DNA sequence, resulting in mutation, insertion, and/or deletion of a DNA sequence at the target site(s) in a genome.

### BRIEF DESCRIPTION OF THE DRAWINGS

FIGS. 1A-1F: The predicted secondary structures of the sgRNA listed in Table 3. FIG. 1A: Scaffold V2. FIG. 1B: Scaffold V2.1. FIG. 1C: Scaffold V2.2. FIG. 1D: Scaffold V2.3. FIG. 1E: Scaffold V2.4. FIG. 1F: Scaffold V2.5.

FIG. 2: OMNI-103 editing activity in Hela cells with different sgRNA scaffolds (Table 3). Hela cells were transfected with OMNI-103 and sgRNA plasmids targeting TRAC-s91 or PDCD-s40. Editing activity was calculated based on next generation sequencing results (bars), and transfection efficiency was based on FACS analysis of the mCherry expression. Presented are the average and standard deviation of three technical replicates.

FIG. 3: Activity in U20S. U2OS cells were electroporated with OMNI-103 and sgRNA (RNP) targeting TRACs35 and B2Ms12. Editing activity was calculated based on next generation sequencing (NGS) results. Presented are the average and standard deviation of three technical replicates.

FIG. 4: Activity in primary T cells. Primary T cells were isolated from PBMCs and activated according to manufacturer's protocol (Miltenyi #130-096-535, #130-091-441). Activated T cells were electroporated with OMNI-103 and sgRNAs (RNPs) targeting TRAC-s35 and B2M-s12. After eight (8) days, cells were measured by flow cytometry for TCR and B2M expression level. For the analysis, only live and CD3-positive cells were counted. The results presented are representative and are one of three T cell donors which all showed similar results.

FIG. 5: T cell activation assay. Donor sample cells used in cleavage activity assay were activated with beads for 72 h and displayed an 85% primary T cell activation rate as measured by FACS (CD3⁺CD25⁺ cells).

FIG. 6: Representative example of an RNA scaffold. An example RNA scaffold portion comprises a crRNA portion linked by a tetraloop to a tracrRNA portion. The crRNA portion comprises a crRNA repeat sequence. The tracrRNA portion comprises a tracrRNA anti-repeat sequence and additional tracrRNA sections. The RNA molecule may further comprise a guide sequence portion (i.e. an RNA spacer) linked to the crRNA repeat sequence, such that the RNA molecule functions as a single-guide RNA molecule.

### DETAILED DESCRIPTION

The invention provides a composition comprising a non-naturally occurring RNA molecule, the RNA molecule comprising a crRNA repeat sequence portion and a guide sequence portion, wherein the RNA molecule forms a complex with and targets an OMNI-103 protein to a DNA target site in the presence of a tracrRNA sequence, wherein the tracrRNA sequence is encoded by a tracrRNA portion of the RNA molecule or a tracrRNA portion of a second RNA molecule. In some embodiments, the OMNI-103 is a nuclease, a nickase, or a catalytically dead nuclease. In some embodiments, the OMNI-103 is encoded by SEQ ID NO: 1, or is a catalytic variant thereof. In some embodiments, the OMNI-103 is a nuclease capable of forming a double-stranded DNA break. In some embodiments, the OMNI-103 is a nickase capable of forming a DNA break in only a single strand of a double-stranded DNA.

In some embodiments, the crRNA repeat sequence portion is up to 17 nucleotides in length, preferably 14-17 nucleotides in length.

In some embodiments, the crRNA repeat sequence portion has at least 60-70%, 71-80%, 81-90%, 91-95%, or 96-99% sequence identity to SEQ ID NOs: 22 or 23.

In some embodiments, the crRNA repeat sequence portion has at least 95% sequence identity to any one of SEQ ID NOs: 22 or 23.

In some embodiments, the crRNA repeat sequence is other than SEQ ID NO: 23.

In some embodiments, the RNA molecule comprising the crRNA repeat sequence portion and the guide sequence portion further comprises the tracrRNA portion.

In some embodiments, the crRNA repeat sequence portion is covalently linked to the tracrRNA portion by a polynucleotide linker portion.

In some embodiments, the composition comprises a second RNA molecule comprising the tracrRNA portion.

In some embodiments, the OMNI-103 nuclease has at least 95% sequence identity to the amino acid sequence of SEQ ID NO: 1. In some embodiments, the OMNI-103 nuclease is a nickase having at least 95% sequence identity to the amino acid sequence of SEQ ID NO: 1 and an amino acid substitution in a position selected from: D12, E776, H988, D991, D856, H857, and N880. In some embodiments, the OMNI-103 nuclease is a catalytically dead nuclease having at least 95% sequence identity to the amino acid sequence of SEQ ID NO: 1, at least one amino acid substitution in position selected from: D12, E776, H988, and D991, and at least one amino acid substitution in a position selected from: D856, H857, and N880.

In some embodiments, the guide sequence portion is 17-30 nucleotides in length, preferably 22 nucleotides in length.

The invention also provides a composition comprising a non-naturally occurring RNA molecule, or a polynucleotide molecule encoding the RNA molecule, the RNA molecule comprising a tracrRNA portion, wherein the RNA molecule forms a complex with and targets an OMNI-103 nuclease to a DNA target site in the presence of a crRNA repeat sequence portion and a guide sequence portion, wherein the crRNA repeat sequence portion and the guide sequence portion are encoded by the RNA molecule or a second RNA molecule.

In some embodiments, the tracrRNA portion is less than 85 nucleotides in length, preferably 84-80, 79-75, 74-70, 69-65, or 64-60 nucleotides in length.

In some embodiments, the tracrRNA portion has at least 30-40%, 41-50%, 51-60%, 61-70%, 71-80%, 81-90%, 91-95%, or 96-99% sequence identity to the tracrRNA portion of any one of SEQ ID NOs: 17-21.

In some embodiments, the tracrRNA portion has at least 95% sequence identity to the tracrRNA portions of any one of SEQ ID NOs: 17-21.

In some embodiments, the tracrRNA portion is other than the tracr portion of SEQ ID NO: 4 or 5.

In some embodiments, the tracrRNA portion comprises a tracrRNA anti-repeat sequence portion that is up to 19 nucleotides in length, preferably 16-19 nucleotides in length.

In some embodiments, the tracrRNA portion comprises a tracrRNA anti-repeat sequence portion that has at least 60-70%, 71-80%, 81-90%, 91-95%, or 96-99% sequence identity to any one of SEQ ID NOs: 24 or 25.

In some embodiments, the tracrRNA portion comprises a tracrRNA anti-repeat sequence portion that has at least 95% sequence identity to any one of SEQ ID NOs: 24 or 25.

In some embodiments, the tracrRNA portion comprises a tracrRNA anti-repeat sequence portion having a sequence other than SEQ ID NO: 25.

In some embodiments, the RNA molecule comprises a tracrRNA portion and further comprises a crRNA repeat sequence portion and a guide sequence portion.

In some embodiments, the tracrRNA portion is covalently linked to the crRNA repeat sequence by a polynucleotide linker portion.

In some embodiments, the polynucleotide linker portion is 4-10 nucleotides in length.

In some embodiments, the polynucleotide linker has a sequence of GAAA.

In some embodiments, the composition further comprises a second RNA molecule comprising a crRNA repeat sequence portion and a guide sequence portion.

In some embodiments, the OMNI-103 nuclease is at least 95% sequence identity to the amino acid sequence of SEQ ID NO: 1.

In some embodiments, the guide sequence portion is 17-30 nucleotides in length, preferably 22 nucleotides in length.

The invention also provides a composition comprising a non-naturally occurring RNA molecule, or a polynucleotide molecule encoding the RNA molecule, the RNA molecule comprising an RNA scaffold portion, the RNA scaffold portion having the structure:
crRNA repeat sequence portion-tracrRNA portion;
wherein the RNA scaffold portion forms a complex with and targets an OMNI-103 CRISPR protein to a DNA target site having complimentarity to a guide sequence portion of the RNA molecule.

In some embodiments, the OMNI-103 is a nuclease, a nickase, or a catalytically dead nuclease. In some embodiments, the OMNI-103 is encoded by SEQ ID NO: 1, or is a catalytic variant thereof. In some embodiments, the OMNI-103 is a nuclease capable of forming a double-stranded DNA break. In some embodiments, the OMNI-103 is a nickase capable of forming a DNA break in only a single strand of a double-stranded DNA.

In some embodiments, the OMNI-103 nuclease has at least 95% sequence identity to the amino acid sequence of SEQ ID NO: 1.

In some embodiments, the RNA scaffold portion is 110-105, 104-100, 99-95, 94-90, 89-85, 84-80, 79-75, or 74-70 nucleotides in length.

In some embodiments, the RNA scaffold portion is 107, 101, 95, 85, or 79 nucleotides in length.

In some embodiments, the RNA scaffold portion has at least 30%, at least 40%, at least 50%, at least 60%, at least 70%, at least 80%, at least 85%, at least 90%, or at least 95% sequence identity to any one of SEQ ID NOs: 17-21.

In some embodiments, the crRNA repeat sequence portion is up to 17 nucleotides in length, preferably 14-17 nucleotides in length.

In some embodiments, the crRNA repeat sequence portion has at least 60-70%, 71-80%, 81-90%, 91-95%, or 96-99% sequence identity to SEQ ID NOs: 22 or 23.

In some embodiments, the crRNA repeat sequence portion has at least 95% sequence identity to any one of SEQ ID NOs: 22 or 23.

In some embodiments, the crRNA repeat sequence is other than SEQ ID NO: 23.

In some embodiments, the tracrRNA portion is less than 85 nucleotides in length, preferably 84-80, 79-75, 74-70, 69-65, or 64-60 nucleotides in length.

In some embodiments, the tracrRNA portion has at least 30-40%, 41-50%, 51-60%, 61-70%, 71-80%, 81-90%, 91-95%, or 96-99% sequence identity to the tracrRNA portion of any one of SEQ ID NOs: 17-21.

In some embodiments, the tracrRNA portion has at least 95% sequence identity to the tracrRNA portions of any one of SEQ ID NOs: 17-21.

In some embodiments, the tracrRNA portion is other than the tracrRNA portion of SEQ ID NO: 4 or 5.

In some embodiments, the RNA scaffold portion further comprises a linker portion between the crRNA repeat sequence portion and the tracrRNA portion such that the RNA scaffold has the structure:
crRNA repeat sequence portion-linker portion-tracrRNA portion.

In some embodiments, the tracrRNA portion comprises a tracrRNA anti-repeat sequence portion, wherein the crRNA repeat sequence and the tracrRNA anti-repeat sequence portion are covalently linked by the linker portion.

In some embodiments, the linker portion is a polynucleotide linker that is 4-10 nucleotides in length.

In some embodiments, the polynucleotide linker has a sequence of GAAA.

In some embodiments, the tracrRNA portion comprises a tracrRNA anti-repeat sequence portion that is up to 19 nucleotides in length, preferably 16-19 nucleotides in length.

In some embodiments, the tracrRNA portion comprises a tracrRNA anti-repeat sequence portion that has at least 60-70%, 71-80%, 81-90%, 91-95%, or 96-99% sequence identity to any one of SEQ ID NOs: 24 or 25.

In some embodiments, the tracrRNA portion comprises a tracrRNA anti-repeat sequence portion that has at least 95% sequence identity to any one of SEQ ID NOs: 24 or 25.

In some embodiments, the tracrRNA anti-repeat sequence is other than SEQ ID NO: 25.

In some embodiments, the tracrRNA portion comprises a first section of nucleotides linked to the tracrRNA anti-repeat portion, and the first section of nucleotides has at least 95% sequence identity to any one of SEQ ID NOs: 26-28.

In some embodiments, the tracrRNA portion comprises a second section of nucleotides linked to a first section of nucleotides, and the second section of nucleotides has at least 95% sequence identity to any one of SEQ ID NOs: 29-32.

In some embodiments, the RNA scaffold portion has at least 95% identity to the nucleotide sequence of any one of SEQ ID NOs: 17-21.

In some embodiments, the RNA scaffold portion has a predicted structure of any one of the V2, V2.1, V2.2, V2.3, V2.4, or V2.5 RNA scaffolds.

In some embodiments, the RNA scaffold portion has a sequence other than SEQ ID NO: 4 or 5.

In some embodiments, a guide sequence portion is covalently linked to the crRNA repeat sequence portion of the RNA molecule, forming a single-guide RNA molecule having a structure:
guide sequence portion-crRNA repeat sequence portion-tracrRNA portion.

In some embodiments, the guide sequence portion is 17-30 nucleotides, more preferably 20-23 nucleotides, more preferably 22 nucleotides in length.

In some embodiments, the composition further comprises an OMNI-103 CRISPR nuclease, wherein the OMNI-103 CRISPR nuclease has at least 95% identity to the amino acid sequence of SEQ ID NO: 1.

In some embodiments, the RNA molecule is formed by in vitro transcription (IVT) or solid-phase artificial oligonucleotide synthesis.

In some embodiments, the RNA molecule comprises modified nucleotides.

In some embodiments, the RNA molecule comprises a sequence of any one of SEQ ID NOs: 17-21. For example, the RNA molecule may be a sgRNA molecule having a scaffold with a sequence of any one of SEQ ID NOs: 17-21.

In some embodiments, the RNA molecule comprises a sequence of SEQ ID NO: 18.

In some embodiments, the RNA molecule comprises a sequence of SEQ ID NO: 19.

In some embodiments, the RNA molecule consists of a guide sequence portion and a sequence of any one of SEQ ID NOs: 17-21. For example, in some embodiments the RNA molecule is a sgRNA molecule which consists of a guide sequence portion and a scaffold, wherein the scaffold has a sequence of any one of SEQ ID NOs: 17-21.

The invention also provides a polynucleotide molecule encoding the RNA molecule of any one of the above embodiments.

The invention also provides a method of modifying a nucleotide sequence at a DNA target site in a cell-free system or a genome of a cell comprising introducing into the system or cell the composition of any one of claims 1-57 and a CRISPR nuclease having at least 95% sequence identity to the amino acid sequence of SEQ ID NO: 1.

In some embodiments, the cell is a eukaryotic cell or a prokaryotic cell.

In some embodiments, the eukaryotic cell is a human cell or a plant cell.

The invention also provides a kit for modifying a nucleotide sequence at a DNA target site in a cell-free system or a genome of a cell comprising introducing into the system or cell the composition of any one of the above embodiments, a CRISPR nuclease having at least 95% sequence identity to the amino acid sequence of SEQ ID NO: 1, and instructions for delivering the RNA molecule and the CRISPR nuclease to the cell.

In embodiments of the present invention, the non-naturally occurring RNA molecule comprises a “spacer” or “guide sequence” portion. The “spacer portion” or “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, 24, 25, 26, 27, 28, 29, or 30 nucleotides in length, or approximately 17-30, 17-29, 17-28, 17-27, 17-26, 17-25, 17-24, 18-22, 19-22, 18-20, 17-20, or 21-22 nucleotides in length. Preferably, the entire length of the guide sequence portion is fully complementary to the DNA sequence being targeted along the length of the guide sequence portion. The guide sequence portion may be part of an RNA molecule having a “scaffold portion” that can form a complex with and activate a CRISPR nuclease, with the guide sequence portion of the RNA molecule serving as the DNA targeting portion of the CRISPR complex. When the RNA molecule having a scaffold portion and a guide sequence portion is present contemporaneously with the CRISPR molecule, the RNA molecule is capable of targeting the CRISPR nuclease to the specific target DNA sequence. Each possibility represents a separate embodiment. The RNA molecule spacer portion can be custom designed to target any desired sequence.

In an embodiment, the nuclease-binding RNA nucleotide sequence and the DNA-targeting RNA nucleotide sequence (e.g. spacer or guide sequence portion) are on a single-guide RNA molecule (sgRNA), wherein the sgRNA molecule can form a complex with the OMNI-103 CRISPR nuclease and serve as the DNA targeting module.

In an embodiment, the nuclease-binding RNA nucleotide sequence is on a first RNA molecule and the DNA-targeting RNA nucleotide sequence is on a second RNA molecule, and the first and second RNA molecules interact by base-pairing and complex with the CRISPR nuclease to serve as the targeting module.

According to some aspects of the invention, the disclosed methods comprise a method of modifying a nucleotide sequence at a target site in a cell-free system or the genome of a cell comprising introducing into the cell the composition of any one of the embodiments described herein.

This invention also provides use of any of the compositions or methods of the invention for modifying a nucleotide sequence at a DNA target site in a cell.

This invention provides a method of modifying a nucleotide sequence at a target site in the genome of a eukaryotic cell.

This invention provides a method of modifying a nucleotide sequence at a target site in the genome of a mammalian cell. In some embodiments, the mammalian cell is a human cell.

This invention provides a method of modifying a nucleotide sequence at a target site in the genome of a plant cell.

In some embodiments, the method is performed ex vivo. In some embodiments, the method is performed in vivo. In some embodiments, some steps of the method are performed ex vivo and some steps are performed in vivo. In some embodiments the mammalian cell is a human cell.

This invention also provides a modified cell or cells obtained by any of the methods described herein. In an embodiment these modified cell or cells are capable of giving rise to progeny cells. In an embodiment these modified cell or cells are capable of giving rise to progeny cells after engraftment.

This invention also provides a composition comprising these modified cells and a pharmaceutically acceptable carrier. Also provided is an in vitro or ex vivo method of preparing this, comprising mixing the cells with the pharmaceutically acceptable carrier.

The nuclease-RNA guide complex described herein can target a desired DNA target sequence via a guide RNA molecule. The nuclease-guide complex will also carry any molecule attached to the complex to the target site. Thus, this disclosure also contemplates fusion proteins comprising a CRISPR nuclease and a DNA modifying domain (e.g., a deaminase, a nuclease, a nickase, a recombinase, a methyltransferase, a methylase, an acetylase, an acetyltransferase, a transcriptional activator, or a transcriptional repressor domain), as well as the use of such fusion proteins in correcting mutations in a genome (e.g., the genome of a human subject) that are associated with disease, or generating mutations in a genome (e.g., the human genome) to decrease or prevent expression of a gene.

In some embodiments, any of the CRISPR nucleases provided herein may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

Thus, any one of the CRISPR nucleases described herein, or a version of the nuclease modified to be a nickase (i.e. having single-strand DNA break activity) or a catalytically dead nuclease (i.e. unable to create any DNA strand break), may be fused (e.g. directly fused or fused via a linker) to another DNA modulating or DNA modifying enzyme, including, but not limited to, base editors such as a deaminase, a reverse transcriptase (e.g. for use in prime editing, see Anzaolone et al. (2019)), an enzyme that modifies the methylation state of DNA (e.g. a methyltransferase), or a modifier of histones (e.g. a histone acetyl transferase). Indeed, the OMNI-103 nucleases, nickases, inactive nucleases described herein may be fused to a DNA modifying enzyme or an effector domain thereof. Examples of DNA modifiers include but are not limited to: a deaminase, a nuclease, a nickase, a recombinase, a methyltransferase, a methylase, an acetylase, an acetyltransferase, a reverse transcriptase, an helicase, an integrase, a ligase, a transposase, a demethylase, a phosphatase, a transcriptional activator, or a transcriptional repressor. In some embodiments, any of the CRISPR nucleases provided herein are fused to a protein that has an enzymatic activity. In some embodiments, the enzymatic activity modifies a target DNA molecule. In some embodiments, the CRISPR nucleases described herein or fusion proteins thereof, may be used to correct or generate one or more mutations in a gene associated with disease, or to increase, correct, decrease or prevent expression of a gene.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, cell dysregulation and oncology diseases and disorders, inflammation and immune-related diseases and disorders, metabolic, liver, kidney and protein diseases and disorders, muscular and skeletal diseases and disorders, dermatological diseases and disorders, neurological and neuronal diseases and disorders, corneal diseases and disorders, retinal diseases and disorders, and ocular diseases and disorders.

### Diseases and Therapies

Certain embodiments of the invention target a nuclease to a specific genetic locus associated with a disease or disorder as a form of gene editing, method of treatment, or therapy. For example, to induce editing or knockout of a gene, a composition disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-103 nuclease to a target site is designed to contain a spacer region complementary to a region neighboring the OMNI-103 PAM sequence “NGG.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects. For example, a guide RNA molecule designed to target OMNI-103 nuclease may be designed to contain a 22-nucleotide spacer for high on-target cleavage activity.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT International Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated gene may be studied, altered, or targeted by any one of the nucleases disclosed herein to treat the disease caused by the disease-associated gene. Non-limiting examples are those listed in U.S. Publication No. 2018/0282762A1 and European Patent No. EP3079726B1.

TABLE A
Diseases, Disorders and their associated genes

| DISEASE/DISORDERS | GENE(S) |
|---|---|
| Neoplasia | PTEN; ATM; ATR; EGFR; ERBB2; ERBB3; ERBB4; Notch1; |
| | Notch2; Notch3; Notch4; AKT; AKT2; AKT3; HIF; HIF1a; |
| | HIF3a; Met; HRG; Bcl2; PPAR alpha; PPAR gamma; WT1 |
| | (Wilms Tumor); FGF Receptor Family members (5 members: 1, |
| | 2, 3, 4, 5); CDKN2a; APC; RB (retinoblastoma); MEN1; VHL; |
| | BRCA1; BRCA2; AR (Androgen Receptor); TSG101; IGF; IGF |
| | Receptor; Igf1 (4 variants); gf2 (3 variants); Igf 1 Receptor; Igf 2 |
| | Receptor; Bax; Bcl2; caspases family (9 members: 1, 2, 3, 4, 6, |
| | 7, 8, 9, 12); Kras; Apc |
| Age-related Macular | Abcr; Ccl2; Cc2; cp (ceruloplasmin); Timp3; cathepsinD; Vldlr; |
| Degeneration | Ccr2 |
| Schizophrenia | Neuregulin1 (Nrg1); Erb4 (receptor for Neuregulin); |
| | Complexin1 (Cp1x1); Tph1 Tryptophan hydroxylase; Tph2 |
| | Tryptophan hydroxylase 2; Neurexin 1; GSK3; GSK3a; GSK3b |
| Neurological, Neuro | 5-HTT (S1c6a4); COMT; DRD (Drd1a); SLC6A3; DAOA; |
| degenerative, and | DTNBP1; Dao (Dao1) |
| Movement Disorders | |
| Trinucleotide Repeat | HTT (Huntington's Dx); SBMA/SMAX1/AR (Kennedy's Dx); |
| Disorders | FXN/X25 (Friedrich's Ataxia); ATX3 (Machado-Joseph's Dx); |
| | ATXN1 and ATXN2 (spinocerebellar ataxias); DMPK |
| | (myotonic dystrophy); Atrophin-1 and Atn1 (DRPLA Dx); CBP |
| | (Creb-BP - global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); |
| Disorders | PEN-2 |
| Others | Nos1; Parp1; Nat1; Nat2 |
| Prion related disorders | Prp |
| ALS | SOD1; ALS2; STEX; FUS; TARDBP; VEGF (VEGF-a; VEGF- |
| | b; VEGF-c) |
| Addiction | Prkce (alcohol); Drd2; Drd4; ABAT (alcohol); GRIA2; Grm5; |
| | Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, Aquaporin |
| | 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); CTLA4; |
| | Cx3cl1 |
| Parkinson's Disease | x-Synuclein; DJ-1; LRRK2; Parkin; PINK1 |

TABLE B
Diseases, Disorders and their associated genes

| DISEASE CATEGORY | DISEASE AND ASSOCIATED GENES |
|---|---|
| Blood and coagulation | Anemia (CDAN1, CDA1, RPS19, DBA, PKLR, PK1, NT5C3, |
| diseases and disorders | UMPH1, PSN1, RHAG, RH50A, NRAMP2, SPTB, ALAS2, |
| | ANH1, ASB, ABCB7, ABC7, ASAT); Bare lymphocyte |
| | syndrome (TAPBP, TPSN, TAP2, ABCB3, PSF2, RING11, |
| | MHC2TA, C2TA, RFX5, RFXAP, RFX5), Bleeding disorders |
| | (TBXA2R, P2RX1, P2X1); Factor H and factor H-like 1 (HF1, |
| | CFH, HUS); Factor V and factor VIII (MCFD2); Factor VII |
| | deficiency (F7); Factor X deficiency (F10); Factor XI deficiency |
| | (F11); Factor XII deficiency (F12, HAF); Factor XIIIA |
| | deficiency (F13A1, F13A); Factor XIIIB deficiency (F13B); |
| | Fanconi anemia (FANCA, FACA, FA1, FA, FAA, FAAP95, |
| | FAAP90, FLJ34064, FANCB, FANCC, FACC, BRCA2, |
| | FANCD1, FANCD2, FANCD, FACD, FAD, FANCE, FACE, |
| | FANCF, XRCC9, FANCG, BRIP1, BACH1, FANCJ, PHF9, |
| | FANCL, FANCM, KIAA1596); Hemophagocytic |
| | lymphohistiocytosis disorders (PRF1, HPLH2, UNC13D, |
| | MUNC13-4, HPLH3, HLH3, FHL3); Hemophilia A (F8, F8C, |
| | HEMA); Hemophilia B (F9, HEMB), Hemorrhagic disorders |
| | (PI, ATT, F5); Leukocyde deficiencies and disorders (ITGB2, |
| | CD18, LCAMB, LAD, EIF2B1, EIF2BA, EIF2B2, EIF2B3, |
| | EIF2B5, LVWM, CACH, CLE, EIF2B4); Sickle cell anemia |
| | (HBB); Thalassemia (HBA2, HBB, HBD, LCRB, HBA1) |
| Cell dysregulation and | B-cell non-Hodgkin lymphoma (BCL7A, BCL7); Leukemia |
| oncology diseases and | (TAL1, TCL5, SCL, TAL2, FLT3, NBS1, NBS, ZNFN1A1, |
| disorders | IK1, LYF1, HOXD4, HOX4B, BCR, CML, PHL, ALL, ARNT, |
| | KRAS2, RASK2, GMPS, AF10, ARHGEF12, LARG, |
| | KIAA0382, CALM, CLTH, CEBPA, CEBP, CHIC2, BTL, |
| | FLT3, KIT, PBT, LPP, NPM1, NUP214, D9S46E, CAN, CAIN, |
| | RUNX1, CBFA2, AML1, WHSC1L1, NSD3, FLT3, AF1Q, |
| | NPM1, NUMA1, ZNF145, PLZF, PML, MYL, STAT5B, AF10, |
| | CALM, CLTH, ARL11, ARLTS1, P2RX7, P2X7, BCR, CML, |
| | PHL, ALL, GRAF, NF1, VRNF, WSS, NFNS, PTPN11, |
| | PTP2C, SHP2, NS1, BCL2, CCND1, PRAD1, BCL1, TCRA, |
| | GATA1, GF1, ERYF1, NFE1, ABL1, NQO1, DIA4, NMOR1, |
| | NUP214, D9S46E, CAN, CAIN) |
| Inflammation and immune | AIDS (KIR3DL1, NKAT3, NKB1, AMB11, KIR3DS1, IFNG, |
| related diseases and | CXCL12, SDF1); Autoimmune lymphoproliferative syndrome |
| disorders | (TNFRSF6, APT1, FAS, CD95, ALPS1A); Combined |
| | immunodeficiency, (IL2RG, SCIDX1, SCIDX, IMD4); HIV-1 |
| | (CCL5, SCYA5, D17S136E, TCP228), HIV susceptibility or |
| | infection (IL10, CSIF, CMKBR2, CCR2, CMKBR5, CCCKR5 |
| | (CCR5)); Immunodeficiencies (CD3E, CD3G, AICDA, AID, |
| | HIGM2, TNFRSF5, CD40, UNG, DGU, HIGM4, TNFSF5, |
| | CD40LG, HIGM1, IGM, FOXP3, IPEX, AIID, XPID, PIDX, |
| | TNFRSF14B, TACI); Inflammation (IL-10, IL-1 (IL-1a, IL-1b), |
| | IL-13, IL-17 (IL-17a (CTLA8), IL-17b, IL-17c, IL- 17d, IL- |
| | 17f), II-23, Cx3cr1, ptpn22, TNFa, NOD2/CARD15 for IBD, |
| | IL-6, IL-12 (IL-12a, IL-12b), CTLA4, Cx3cl1); Severe |
| | combined immunodeficiencies (SCIDs)(JAK3, JAKL, |
| | DCLRE1C, ARTEMIS, SCIDA, RAG1, RAG2, ADA, PTPRC, |
| | CD45, LCA, IL7R, CD3D, T3D, IL2RG, SCIDX1, SCIDX, |
| | IMD4) |
| Metabolic, liver, kidney | Amyloid neuropathy (TTR, PALB); Amyloidosis (APOA1, |
| and protein diseases and | APP, AAA, CVAP, AD1, GSN, FGA, LYZ, TTR, PALB); |
| disorders | Cirrhosis (KRT18, KRT8, CIRH1A, NAIC, TEX292, |
| | KIAA1988); Cystic fibrosis (CFTR, ABCC7, CF, MRP7); |
| | Glycogen storage diseases (SLC2A2, GLUT2, G6PC, G6PT, |
| | G6PT1, GAA, LAMP2, LAMPB, AGL, GDE, GBE1, GYS2, |
| | PYGL, PFKM); Hepatic adenoma, 142330 (TCF1, HNF1A, |
| | MODY3), Hepatic failure, early onset, and neurologic disorder |
| | (SCOD1, SCO1), Hepatic lipase deficiency (LIPC), |
| | Hepatoblastoma, cancer and carcinomas (CTNNB1, PDGFRL, |
| | PDGRL, PRLTS, AXIN1, AXIN, CTNNB1, TP53, P53, LFS1, |
| | IGF2R, MPRI, MET, CASP8, MCH5; Medullary cystic kidney |
| | disease (UMOD, HNFJ, FJHN, MCKD2, ADMCKD2); |
| | Phenylketonuria (PAH, PKU1, QDPR, DHPR, PTS); Polycystic |
| | kidney and hepatic disease (FCYT, PKHD1, ARPKD, PKD1, |
| | PKD2, PKD4, PKDTS, PRKCSH, G19P1, PCLD, SEC63) |
| Muscular/Skeletal | Becker muscular dystrophy (DMD, BMD, MYF6), Duchenne |
| diseases and disorders | Muscular Dystrophy (DMD, BMD); Emery-Dreifuss muscular |
| | dystrophy (LMNA, LMN1, EMD2, FPLD, CMD1A, HGPS, |
| | LGMD1B, LMNA, LMN1, EMD2, FPLD, CMD1A); |
| | Facioscapulohumeral muscular dystrophy (FSHMD1A, |
| | FSHD1A); Muscular dystrophy (FKRP, MDC1C, LGMD2I, |
| | LAMA2, LAMM, LARGE, KIAA0609, MDC1D, FCMD, |
| | TTID, MYOT, CAPN3, CANP3, DYSF, LGMD2B, SGCG, |
| | LGMD2C, DMDA1, SCG3, SGCA, ADL, DAG2, LGMD2D, |
| | DMDA2, SGCB, LGMD2E, SGCD, SGD, LGMD2F, CMD1L, |
| | TCAP, LGMD2G, CMD1N, TRIM32, HT2A, LGMD2H, |
| | FKRP, MDC1C, LGMD2I, TTN, CMD1G, TMD, LGMD2J, |
| | POMT1, CAV3, LGMD1C, SEPN1, SELN, RSMD1, PLEC1, |
| | PLTN, EBS1); Osteopetrosis (LRP5, BMND1, LRP7, LR3, |
| | OPPG, VBCH2, CLCN7, CLC7, OPTA2, OSTM1, GL, |
| | TCIRG1, TIRC7, OC116, OPTB1); Muscular atrophy (VAPB, |
| | VAPC, ALS8, SMN1, SMA1, SMA2, SMA3, SMA4, BSCL2, |
| | SPG17, GARS, SMAD1, CMT2D, HEXB, IGHMBP2, |
| | SMUBP2, CATF1, SMARD1) |
| Dermatological diseases | Albinisim (TYR, OCA2, TYRP1, SLC45A2, LYST), |
| and disorders | Ectodermal dysplasias (EDAR, EDARADD, WNT10A), Ehlers- |
| | Danlos syndrome (COL5A1, COL5A2, COL1A1, COL1A2, |
| | COL3A1, TNXB, ADAMTS2, PLOD1, FKBP14), Ichthyosis- |
| | associated disorders (FLG, STS, TGM1, ALOXE3/ALOX12B, |
| | KRT1, KRT10, ABCA12, KRT2, GJB2, TGM1, ABCA12, |
| | CYP4F22, ALOXE3, CERS3, NSHDL, EBP, MBTPS2, GJB2, |
| | SPINK5, AGHD5, PHYH, PEX7, ALDH3A2, ERCC2, ERCC3, |
| | GFT2H5, GBA), Incontinentia pigmenti (IKBKG, NEMO), |
| | Tuberous sclerosis (TSC1, TSC2), Premature aging syndromes |
| | (POLR3A, PYCR1, LMNA, POLD1, WRN, DMPK) |
| Neurological and Neuronal | ALS (SOD1, ALS2, STEX, FUS, TARDBP, VEGF (VEGF-a, |
| diseases and disorders | VEGF-b, VEGF-c); Alzheimer disease (APP, AAA, CVAP, |
| | AD1, APOE, AD2, PSEN2, AD4, STM2, APBB2, FE65L1, |
| | NOS3, PLAU, URK, ACE, DCP1, ACE1, MPO, PACIP1, |
| | PAXIP1L, PTIP, A2M, BLMH, BMH, PSEN1, AD3); Autism |
| | (Mecp2, BZRAP1, MDGA2, Sema5A, Neurexin 1, GLO1, |
| | MECP2, RTT, PPMX, MRX16, MRX79, NLGN3, NLGN4, |
| | KIAA1260, AUTSX2); Fragile X Syndrome (FMR2, FXR1, |
| | FXR2, mGLUR5); Huntington's disease and disease like |
| | disorders (HD, IT15, PRNP, PRIP, JPH3, JP3, HDL2, TBP, |
| | SCA17); Parkinson disease (NR4A2, NURR1, NOT, TINUR, |
| | SNCAIP, TBP, SCA17, SNCA, NACP, PARK1, PARK4, DJ1, |
| | PARK7, LRRK2, PARK8, PINK1, PARK6, UCHL1, PARK5, |
| | SNCA, NACP, PARK1, PARK4, PRKN, PARK2, PDJ, DBH, |
| | NDUFV2); Rett syndrome (MECP2, RTT, PPMX, MRX16, |
| | MRX79, CDKL5, STK9, MECP2, RTT, PPMX, MRX16, |
| | MRX79, x-Synuclein, DJ-1); Schizophrenia (Neuregulin1 |
| | (Nrg1), Erb4 (receptor for Neuregulin), Complexin1 (Cplx1), |
| | Tph1 Tryptophan hydroxylase, Tph2, Tryptophan hydroxylase 2, |
| | Neurexin 1, GSK3, GSK3a, GSK3b, 5-HTT (Slc6a4), COMT, |
| | DRD (Drd1a), SLC6A3, DAOA, DTNBP1, Dao (Dao1)); |
| | Secretase Related Disorders (APH-1 (alpha and beta), Presenilin |
| | (Psen1), nicastrin, (Ncstn), PEN-2, Nos1, Parp1, Natl, Nat2); |
| | Trinucleotide Repeat Disorders (HTT (Huntington's Dx), |
| | SBMA/SMAX1/AR (Kennedy's Dx), FXN/X25 (Friedrich's |
| | Ataxia), ATX3 (Machado-Joseph's Dx), ATXN1 and ATXN2 |
| | (spinocerebellar ataxias), DMPK (myotonic dystrophy), |
| | Atrophin-1 and Atn1 (DRPLA Dx), CBP (Creb-BP - global |
| | instability), VLDLR (Alzheimer's), Atxn7, Atxn10) |
| Ocular diseases and | Age-related macular degeneration (Abcr, Ccl2, Cc2, cp |
| disorders | (ceruloplasmin), Timp3, cathepsinD, Vldlr, Ccr2); Cataract |
| | (CRYAA, CRYA1, CRYBB2, CRYB2, PITX3, BFSP2, CP49, |
| | CP47, CRYAA, CRYA1, PAX6, AN2, MGDA, CRYBA1, |
| | CRYB1, CRYGC, CRYG3, CCL, LIM2, MP19, CRYGD, |
| | CRYG4, BFSP2, CP49, CP47, HSF4, CTM, HSF4, CTM, MIP, |
| | AQP0, CRYAB, CRYA2, CTPP2, CRYBB1, CRYGD, CRYG4, |
| | CRYBB2, CRYB2, CRYGC, CRYG3, CCL, CRYAA, CRYA1, |
| | GJA8, CX50, CAE1, GJA3, CX46, CZP3, CAE3, CCM1, CAM, |
| | KRIT1); Corneal clouding and dystrophy (APOA1, TGFBI, |
| | CSD2, CDGG1, CSD, BIGH3, CDG2, TACSTD2, TROP2, |
| | M1S1, VSX1, RINX, PPCD, PPD, KTCN, COL8A2, FECD, |
| | PPCD2, PIP5K3, CFD); Cornea plana congenital (KERA, |
| | CNA2); Glaucoma (MYOC, TIGR, GLC1A, JOAG, GPOA, |
| | OPTN, GLC1E, FIP2, HYPL, NRP, CYP1B1, GLC3A, OPA1, |
| | NTG, NPG, CYP1B1, GLC3A); Leber congenital |
| | amaurosis (CRB1, RP12, CRX, CORD2, CRD, RPGRIP1, |
| | LCA6, CORD9, RPE65, RP20, AIPL1, LCA4, GUCY2D, |
| | GUC2D, LCA1, CORD6, RDH12, LCA3); Macular dystrophy |
| | (ELOVL4, ADMD, STGD2, STGD3, RDS, RP7, PRPH2, |
| | PRPH, AVMD, AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

It is understood that where a numerical range is recited herein, the present invention contemplates each integer between, and including, the upper and lower limits, unless otherwise stated.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “polynucleotide”, “nucleotide”, “nucleotide sequence”, “nucleic acid” and “oligonucleotide” are used interchangeably. They refer to a polymeric form of nucleotides of any length, either deoxyribonueleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, in Irons, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers, A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate. Each of the RNA sequences described herein may comprise one or more nucleotide analogs.

As used herein, the following nucleotide identifiers are used to represent a referenced nucleotide base(s):

| Nucleotide | Base(s) | | | |
|---|---|---|---|---|
| reference | represented | | | |
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of a targeting RNA molecule that can form a complex with a CRISPR nuclease, e.g. via a scaffold portion, with the targeting sequence serving as the targeting portion, e.g. spacer portion, of the CRISPR complex. When the RNA molecule having the targeting sequence is present contemporaneously with the CRISPR nuclease, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferential hybridization of a targeting sequence or a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. Throughout this application, the terms “guide molecule,” “RNA guide molecule,” “guide RNA molecule,” and “gRNA molecule” are synonymous with a molecule comprising a guide sequence portion.

In the context of targeting a DNA sequence that is present in a plurality of cells, it is understood that the targeting encompasses hybridization of the guide sequence portion of the RNA molecule with the sequence in one or more of the cells, and also encompasses hybridization of the RNA molecule with the target sequence in fewer than all of the cells in the plurality of cells. Accordingly, it is understood that where an RNA molecule targets a sequence in a plurality of cells, a complex of the RNA molecule and a CRISPR nuclease is understood to hybridize with the target sequence in one or more of the cells, and also may hybridize with the target sequence in fewer than all of the cells. Accordingly, it is understood that the complex of the RNA molecule and the CRISPR nuclease introduces a double strand break in relation to hybridization with the target sequence in one or more cells and may also introduce a double strand break in relation to hybridization with the target sequence in fewer than all of the cells. As used herein, the term “modified cells” refers to cells in which a double strand break is affected by a complex of an RNA molecule and the CRISPR nuclease as a result of hybridization with the target sequence, i.e. on-target hybridization.

As used herein the term “wild type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion) compared to the OMNI-103 CRISPR nuclease indicated in Table 1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

As used herein the term “amino acid” includes natural and/or unnatural or synthetic amino acids, including glycine and both the D or I, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The term “PAM” as used herein refers to a nucleotide sequence of a target DNA located in proximity to the targeted DNA sequence and recognized by the CRISPR nuclease. The PAM sequence may differ depending on the nuclease identity.

The term “mutation disorder” or “mutation disease” as used herein refers to any disorder or disease that is related to dysfunction of a gene caused by a mutation. A dysfunctional gene manifesting as a mutation disorder contains a mutation in at least one of its alleles and is referred to as a “disease-associated gene.” The mutation may be in any portion of the disease-associated gene, for example, in a regulatory, coding, or non-coding portion. The mutation may be any class of mutation, such as a substitution, insertion, or deletion. The mutation of the disease-associated gene may manifest as a disorder or disease according to the mechanism of any type of mutation, such as a recessive, dominant negative, gain-of-function, loss-of-function, or a mutation leading to haploinsufficiency of a gene product.

A skilled artisan will appreciate that embodiments of the present invention disclose RNA molecules comprising a scaffold portion capable of complexing with an OMNI-103 CRISPR nuclease and activating the OMNI-103 CRISPR nuclease to be targeted to a target DNA site of interest that is adjacent to a protospacer adjacent motif (PAM). The OMNI-103 CRISPR nuclease is targeted to a DNA site of interest by a guide sequence portion (i.e. a RNA spacer) having complementarity to the target DNA site of interest. The nuclease then mediates cleavage of target DNA to create a double-strand break within the protospacer target site.

The term “protein binding sequence” or “nuclease binding sequence” refers to a sequence capable of binding with a CRISPR nuclease to form a CRISPR complex. A skilled artisan will understand that scaffold RNA or a tracrRNA capable of binding with a CRISPR nuclease to form a CRISPR complex comprises a protein or nuclease binding sequence.

An “RNA binding portion” of a CRISPR nuclease refers to a portion of the CRISPR nuclease which may bind to an RNA molecule to form a CRISPR complex, e.g. the nuclease binding sequence of an RNA scaffold portion of a sgRNA. An “activity portion” or “active portion” of a CRISPR nuclease refers to a portion of the CRISPR nuclease which effects a double strand break in a DNA molecule, for example when in complex with a DNA-targeting RNA molecule.

The term “RNA scaffold” or “scaffold” refers to a portion of a non-naturally occurring molecule that comprises a crRNA portion covalently linked to a tracrRNA portion. As used herein, a “crRNA portion” comprises a crRNA repeat sequence. As used herein, a “tracrRNA portion” comprises a tracrRNA anti-repeat sequence. A tracrRNA portion may further comprise additional tracrRNA sequences linked to the tracrRNA anti-repeat sequence. Such sequences may include, but are not limited to, a nexus, hairpin, or other tracrRNA sequences upstream or downstream of a nexus, hairpin, or tracrRNA anti-repeat sequence. Accordingly, a tracrRNA portion of an RNA scaffold comprises an anti-repeat sequence, which is optionally linked to additional tracrRNA sections.

As used herein, an RNA molecule comprising an RNA scaffold portion and an RNA guide sequence portion (or RNA spacer portion) serves as a single-guide RNA (sgRNA) molecule. The RNA scaffold portion of the sgRNA specifically binds and activates an CRISPR nuclease, and the RNA spacer portion of the sgRNA targets CRISPR nuclease to a DNA target site. For example, a sgRNA molecule may be formed by covalent linkage of a guide sequence portion to a crRNA repeat sequence portion of an RNA scaffold.

Accordingly, in embodiments of the present invention, the RNA molecule may be designed as a synthetic fusion of a scaffold portion and a spacer portion, together forming a single guide RNA (sgRNA) capable of binding and targeting an OMNI-103 CRISPR nuclease. See Jinek et al., Science (2012).

Embodiments of the present invention may also form CRISPR complexes utilizing a separate crRNA molecule and a separate tracrRNA molecule. In such embodiments the crRNA molecule may hybridize with the tracrRNA molecule via at least partial hybridization between a crRNA repeat sequence portion of the crRNA molecule and a tracrRNA anti-repeat sequence portion of the tracrRNA molecule. Such partial hybridization may also contain a typical bulge that separates the hybridized RNA nucleotides into an “upper” and “lower” stem. Separate crRNA and tracrRNA molecules may be advantageous in certain applications of the invention described herein.

In embodiments of the present invention a scaffold portion of an RNA molecule may comprise a “nexus” region and/or “hairpin” regions which may further define the structure of the RNA molecule. (See Briner et al., Molecular Cell (2014)).

As used herein, the term “direct repeat sequence” refers to two or more repeats of a specific amino acid sequence or nucleotide sequence.

As used herein, an RNA sequence or molecule capable of “interacting with” or “binding” with a CRISPR nuclease refers to the RNA sequence or molecules ability to form a CRISPR complex with the CRISPR nuclease.

As used herein, the term “operably linked” refers to a relationship (i.e. fusion, hybridization) between two sequences or molecules permitting them to function in their intended manner. In embodiments of the present invention, when an RNA molecule is operably linked to a promoter, both the RNA molecule and the promotor are permitted to function in their intended manner.

As used herein, the term “heterologous promoter” refers to a promoter that does not naturally occur together with the molecule or pathway being promoted.

As used herein, a sequence or molecule has an X % “sequence identity” to another sequence or molecule if X % of nucleotides or amino acids between the sequences of molecules are the same and in the same relative position. For example, a first nucleotide sequence having at least a 95% sequence identity with a second nucleotide sequence will have at least 95% of nucleotides, in the same relative position, identical with the other sequence. As non-limiting example, sequence identity may be determined by creating an alignment of a first nucleotide sequence to a second nucleotide sequence, for example, by applying the Needleman-Wunsch algorithm.

### Delivery

The CRISPR nuclease or CRISPR compositions described herein may include and be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-O-methyl (M), 2′-O-methyl, 3′phosphorothioate (MS) or 2′-O-methyl, 3 'thioPACE (MSP), pseudouridine, and 1-methyl pseudo-uridine. Each possibility represents a separate embodiment of the present invention.

The CRISPR nucleases and/or polynucleotides encoding same described herein, and optionally additional proteins (e.g., ZFPs, TALENs, transcription factors, restriction enzymes) and/or nucleotide molecules such as guide RNA may be delivered to a target cell by any suitable means. The target cell may be any type of cell e.g., eukaryotic or prokaryotic, in any environment e.g., isolated or not, maintained in culture, in vitro, ex vivo, in vivo or in planta.

In some embodiments, the composition to be delivered includes mRNA of the nuclease and RNA of the guide molecule. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. In some embodiments, the composition to be delivered includes mRNA of the nuclease, DNA-targeting RNA and the tracrRNA. In some embodiments, the composition to be delivered includes mRNA of the nuclease, DNA-targeting RNA and the tracrRNA and a donor template. In some embodiments, the composition to be delivered includes the CRISPR nuclease DNA-targeting RNA and the tracrRNA. In some embodiments, the composition to be delivered includes the CRISPR nuclease, DNA-targeting RNA and the tracrRNA and a donor template for gene editing via, for example, homology directed repair.

Any suitable viral vector system may be used to deliver RNA compositions. Conventional viral and non-viral based gene transfer methods can be used to introduce nucleic acids and/or CRISPR nuclease in cells (e.g., mammalian cells, plant cells, etc.) and target tissues. Such methods can also be used to administer nucleic acids encoding and/or CRISPR nuclease protein to cells in vitro. In certain embodiments, nucleic acids and/or CRISPR nuclease are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science (1992); Nabel and Felgner, TIBTECH (1993); Mitani and Caskey, TIBTECH (1993); Dillon, TIBTECH (1993); Miller, Nature (1992); Van Brunt, Biotechnology (1988); Vigne et al., Restorative Neurology and Neuroscience 8:35-36 (1995); Kremer and Perricaudet, British Medical Bulletin (1995); Haddada et al., Current Topics in Microbiology and Immunology (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid: nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Non-viral vectors, such as transposon-based systems e.g., recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam. TM., Lipofectin.TM. and Lipofectamine.TM. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid: nucleic acid complexes, including targeted liposomes such as immunolipid complexes, is well known to one of skill in the art (see, e.g., Crystal, Science (1995); Blaese et al., Cancer Gene Ther. (1995); Behr et al., Bioconjugate Chem. (1994); Remy et al., Bioconjugate Chem. (1994); Gao and Huang, Gene Therapy (1995); Ahmad and Allen, Cancer Res., (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al., Nature Biotechnology (2009)).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, recombinant retroviral, lentivirus, adenoviral, adeno-associated, vaccinia and herpes simplex virus vectors for gene transfer. However, an RNA virus is preferred for delivery of the RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and a donor template. Optionally, the lentivirus includes the nuclease protein, guide RNA. Optionally, the lentivirus includes the nuclease protein, guide RNA and/or a donor template for gene editing via, for example, homology directed repair. Optionally the lentivirus includes mRNA of the nuclease, DNA-targeting RNA, and the tracrRNA. Optionally the lentivirus includes mRNA of the nuclease, DNA-targeting RNA, and the tracrRNA, and a donor template. Optionally, the lentivirus includes the nuclease protein, DNA-targeting RNA, and the tracrRNA. Optionally, the lentivirus includes the nuclease protein, DNA-targeting RNA, and the tracrRNA, and a donor template for gene editing via, for example, homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. In some embodiments, delivery of mRNA in vivo and ex vivo, and RNPs delivery may be utilized.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney, “Culture of Animal Cells, A Manual of Basic Technique and Specialized Applications (6th edition, 2010)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO--S, CHO-K1, CHO-DG44, CHO-DUXB11, CHO-DUKX, CHOKISV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR). Suitable primary cells include peripheral blood mononuclear cells (PBMC), and other blood cell subsets such as, but not limited to, CD4+T cells or CD8+T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in-vitro or can be introduced into a mammal (such as the donor of the cells) where they will engraft in the bone marrow. Methods for differentiating CD34+ cells in vitro into clinically important immune cell types using cytokines such a GM-CSF, IFN-gamma. and TNF-alpha are known (as a non-limiting example see, Inaba et al., J. Exp. Med. (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (as a non-limiting example see Inaba et al., J. Exp. Med. (1992)). Stem cells that have been modified may also be used in some embodiments.

Notably, the compositions described herein may be suitable for genome editing in post-mitotic cells or any cell which is not actively dividing, e.g., arrested cells. Examples of post-mitotic cells which may be edited using a CRISPR nuclease of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### DNA Repair by Homologous Recombination

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3' terminus of a linear molecule and/or self-complementary oligonucleotides are ligated to one or both ends. See, for example, Chang and Wilson, Proc. Natl. Acad. Sci. USA (1987); Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) an RNA molecule comprising a guide sequence to affect a double-strand break in a gene prior to repair and (2) a donor RNA template for repair, the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by recombinant viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPPIR12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al., “Molecular Cloning: A laboratory Manual” (1989); Ausubel, R. M. (Ed.), “Current Protocols in Molecular Biology” Volumes I-III (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells-A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, CT (1994); Mishell and Shiigi (Eds.), “Strategies for Protein Purification and Characterization-A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Experimental Details

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Methods

OMNI-103 sequences of the CRISPR repeat (crRNA), transactivating crRNA (tracrRNA) and nuclease polypeptide were predicted from metagenomic sequence database of environmental samples. The full-length guide scaffold with spacer optimization, the NNRRHY PAM and the activity in mammalian were discussed PCT International Publication No. WO2022/170199 A2 published on Aug. 11, 2022, and the main finding elements can be found at Table 1.

### OMNI-103 Protein Expression

Briefly, The nuclease open reading frame was codon optimized to human (Table 1) and cloned into modified pET9a plasmid with the following elements-SV40 NLS-OMNI-103 ORF (from 2nd amino acid human optimized)-HA tag-SV40 NLS-8 His-tag and the sequence can be found in Table 2. The OMNI-103 construct was expressed in KRX cells (PROMEGA). Cells were grown in TB+0.4% Glycerol with the addition of 6.66 mM rhamnose (26.4 ml from 0.5M stock), and 0.05% glucose (2 ml from 0.5M), and expressed in mid-log phase, after 4 hr by temperature reduction to 20° C. Cells were lysed using chemical lysis and cleared lysate was purified on Ni-NTA resin. Ni-NTA elution fraction was purified on CEX (SO3 fractogel) resin followed by SEC purification on Superdex 200 Increase 10/300 GL, AKTA Pure (GE Healthcare Life Sciences). Fractions containing OMNI103 protein were pooled and concentrated to 30 mg/ml stocks and flash-frozen in liquid nitrogen and stored at −80° C.

Synthetic sgRNA Used

All synthetic sgRNAs of OMNI-103 were synthesized with three 2′-O-methyl 3′-phosphorothioate at the 3′ and 5′ ends (Agilent or Synthego).

### Activity in Mammalian Cell Lines

The ability of OMNI-103 to promote editing with the shorter sgRNA versions was tested on specific genomic locations in human cells (Table 4). For HeLa cells, the OMNI-103-P2A-mCherry expression vector (pmOMNI, Table 2) was transfected together with the sgRNA (pShuttle guide, Table 2, spacer sequence, Table 4).

For U2OS cells, RNPs were assembled by mixing 100 μM nuclease with 120 μM of synthetic guide and 100 μM Cas9 electroporation enhancer (IDT). After 10 minutes of incubation at room-temperature, the RNP complexes were mixed with 200,000 pre-washed U2OS cells and electroporated using Lonza SE Cell Line 4D-Nucleofector™ X Kit with the DN100 program, according to the manufacture's protocol. At 72 h cells were lysed, and their genomic DNA content was used in a PCR reaction that amplified the corresponding putative genomic targets. Amplicons were subjected to NGS and the resulting sequences were then used to calculate the percentage of editing events.

For T cells, RNPs were assembled by mixing 113 μM nuclease and 160 μM of synthetic guide and incubating for 10 minutes at R.T. RNP complexes were mixed with 200,000 primary activated T cells, and electroporated using P3 Primary Cell 4D-Nucleofector™ X Kit, with EH-115 pulse code. After three (3) days and eight (8) days cells were collected, and CD3 and the edited protein expression was measured by flow cytometry.

### Results

### Activity of Short Guides Across Genomic Sites and Cell Types

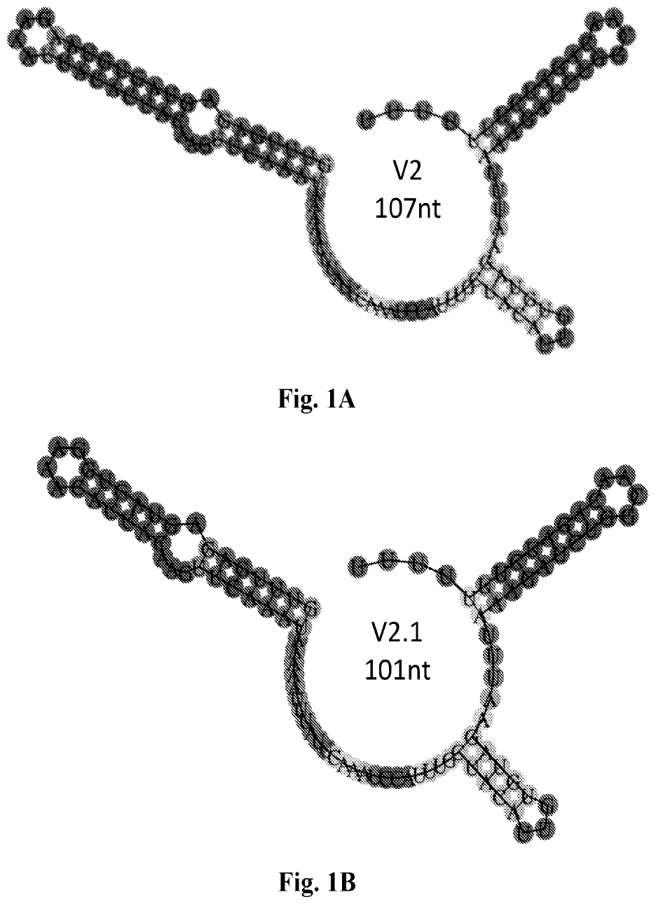

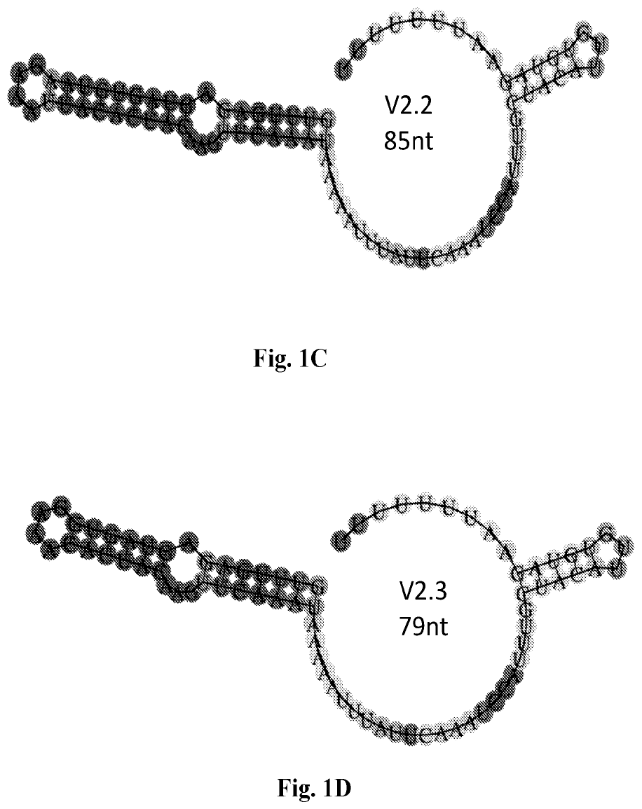

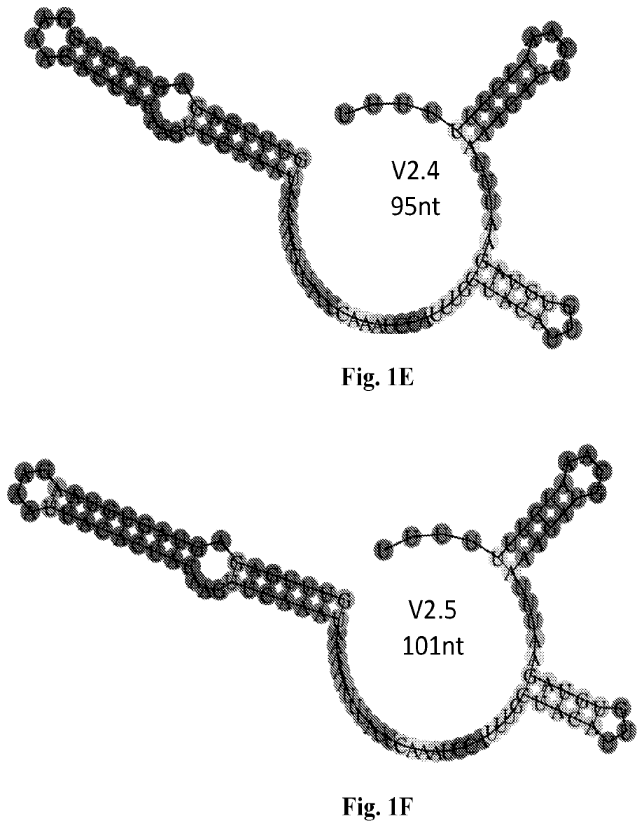

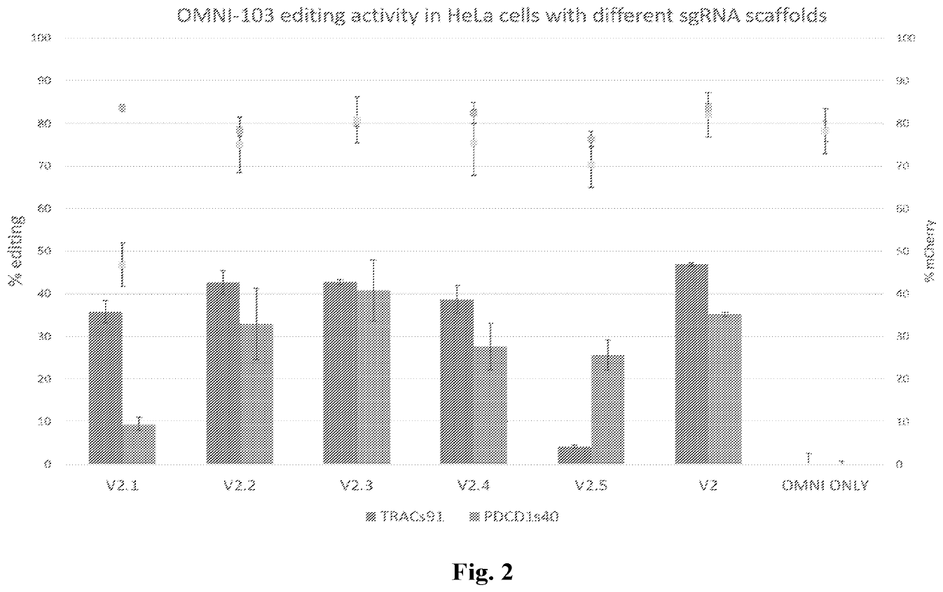

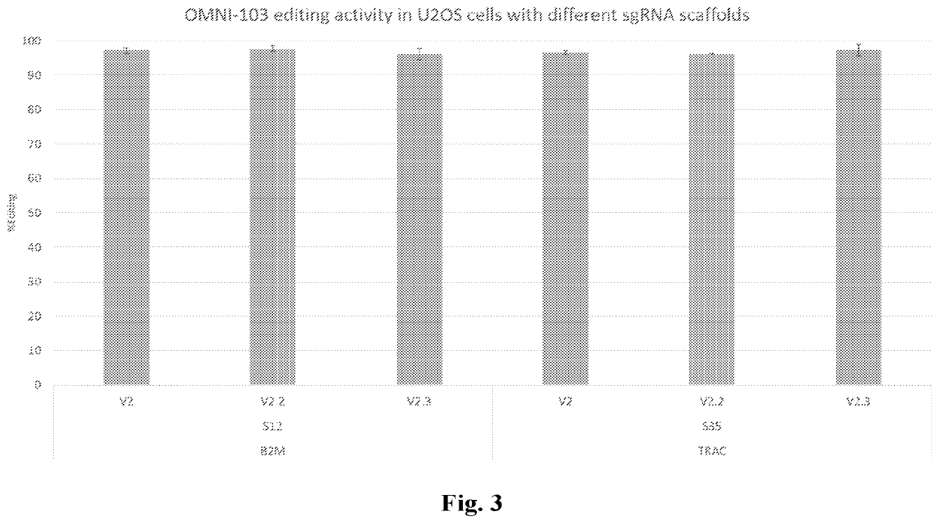

OMNI-103 nuclease activity was optimized for use with shorter sgRNA scaffolds. Five (5) short sgRNA scaffolds were designed based on the ‘V2’ duplex version, which contained up to four deletions around the tetra loop “GAAA” and the terminator region (Table 3, FIGS. 1A-1F). To test the level of activity OMNI-103 displayed with the designed V2 scaffolds, sgRNAs having guide sequence portions of “TRAC-s91” or “PDCD-s40” were transfected into HeLa cells. Editing activity was calculated based on NGS results (FIG. 2). In all cases the designed sgRNA enabled editing activity. The next step was to test OMNI-103 activity as an RNP in U2OS and primary T cells. OMNI-103 was electroporated with sgRNAs having a V2, V2.2 or V2.3 scaffold and having guide sequence portions of “TRAC-s35” or “B2M-s12”. Editing activity was calculated based on NGS results, and as demonstrated the level of OMNI-103 activity was not impaired when used with any of the scaffold variants (FIG. 3). In primary T cells, when the short scaffold variants were utilized, improved activity was demonstrated.

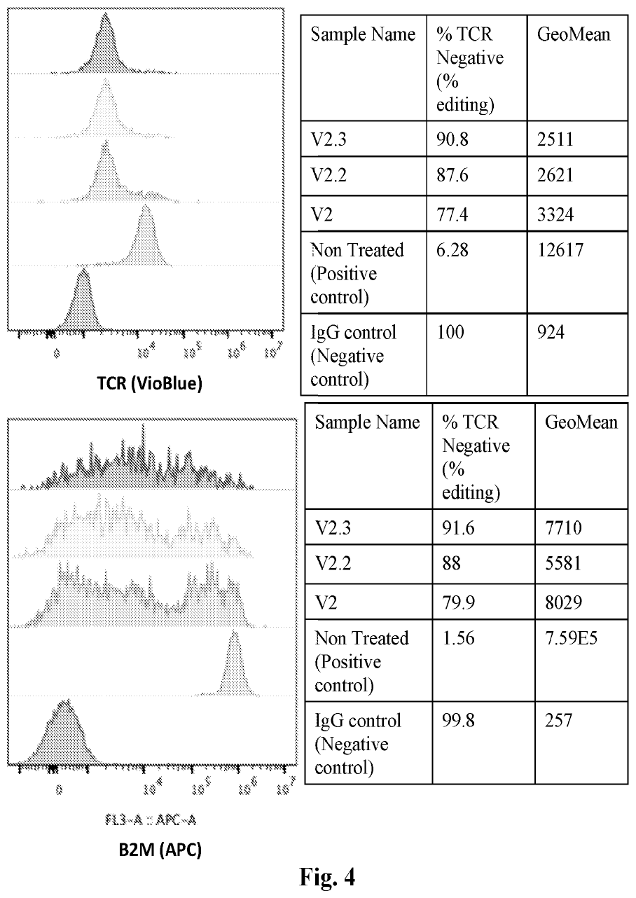

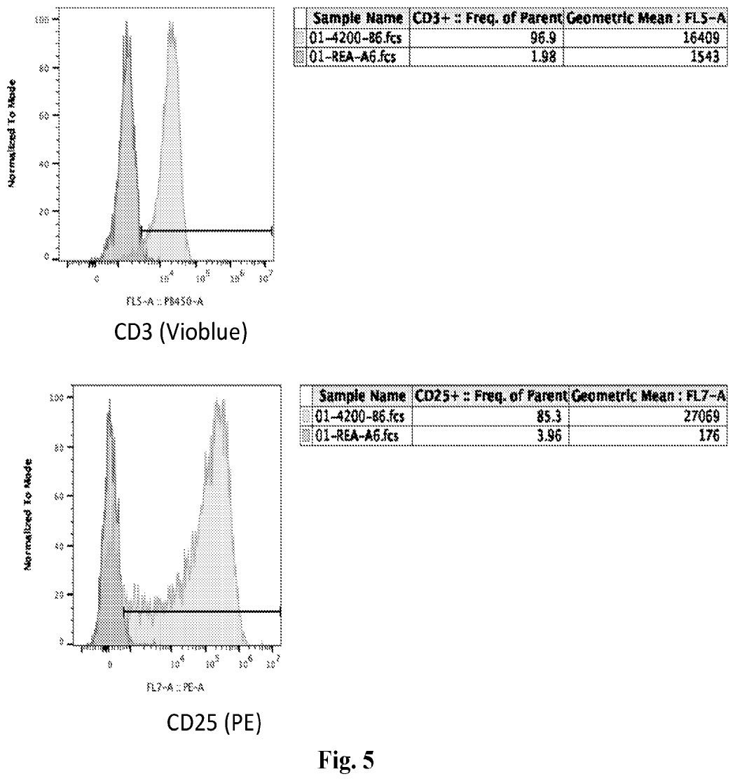

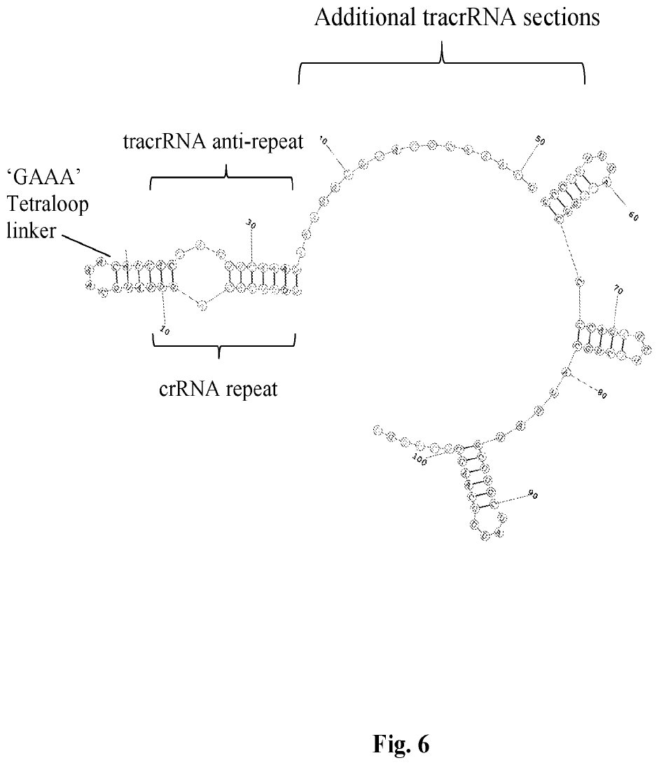

Primary T cells were isolated from human PBMCs, and activated by CD2, CD3 and CD28 according to manufacturer's recommendations (#130-091-441, Miltenyi). After three (3) days, cells were mostly activated (more than 85% CD25-positive cells, see FIG. 5). 200,000 activated primary T cells were electroporated with OMNI-103 nuclease and guide RNA molecules having a V2, V2.2 or V2.3 scaffold and a spacer sequence (i.e. a guide sequence portion) of TRAC-s35 or B2M-s12. Editing after eight (8) days in culture was measured by next generation sequencing (NGS), and the level of protein expression was measured by flow cytometry, which showed a significant reduction in TCR or B2M with the V2 scaffold, and an even larger reduction in protein expression with V2.2 or V2.3 (FIG. 4).

TABLE 1
OMNI-103 genetic elements as discovered in U.S. Provisional
Application Nos. 63/147,166, and 63/214,506

| | |
|---|---|
| Nuclease name | OMNI-103 |
| Protein sequence | SEQ ID NO: 1 |
| DNA sequence of OMNI-103 ORF | SEQ ID NO: 2 |
| Human optimized DNA sequence of | SEQ ID NO: 3 |
| OMNI-103 ORF | |
| OMNI-103 PAM | NNRRHY |
| sgRNA V1 Scaffold | GUUUGAGAGUAGUGUAAgaaaUUACAC |
| | UACAAGUUCAAAUAAAAAUUUAUUC |
| | AAAUCCUUUUGCUACAUUGUGUAGAA |
| | UUUAAAGAUCUGGCAACAGAUCUUUU |
| | UUU (SEQ ID NO: 4) |
| sgRNA V2Scaffold | GUUUGAGAGUAGUGUAAgaaaUUACAC |
| | UACAAGUUCAAAUAAAAAUUUAUUC |
| | AAAUCCAUUUGCUACAUUGUGUAGAA |
| | UUUAAAGAUCUGGCAACAGAUCUUUU |
| | UUU (SEQ ID NO: 5) |

TABLE 2
Plasmid constructs for OMNI-103 Expression and Purification

| Plasmid | Elements | Coding ORF Sequence | DNA Sequence |
|---|---|---|---|
| pET9a: | Expressing OMNI | T7 promoter - SV40 NLS - OMNI | SEQ ID NO: 7 |
| OMNI-103 | polypeptide in the | ORF (Human optimized) - | |
| | bacterial system | HA Tag - SV40 NLS - | |
| | | 8XHisTag - T7 terminator | |
| pShuttle | Expressing OMNI | U6 promoter - T7 promoter - T2 | SEQ ID NO: 8 |
| Guide: | sgRNA in the bacterial | spacer - sgRNA scaffold - T7 | |
| OMNI-103 | and human cell system | terminator | |
| V2 | | | |
| pmOMNI: | Expressing OMNI | CMV promoter - T7 promoter - | SEQ ID NO: 9 |
| OMNI-103 | polypeptide in the | SV40 NLS - OMNI ORF (Human | |
| | human cell system | optimized) - HA - SV40 NLS - | |
| | | P2A - mCherry - BGH poly(A) | |

TABLE 3
OMNI-103 Designed Scaffold Sequences

| Experimental | | | | |
|---|---|---|---|---|
| Name | Full sequence | | | Length |
| V2.1 | GUUUGAGAGUAGUGGAAACACUACAAGUUCAAAUAAAAAUUU | | | 101 |
| | AUUCAAAUCCAUUUGCUACAUUGUGUAGAAUUUAAAGAUCUG | | | |
| | GCAACAGAUCUUUUUUU (SEQ ID NO: 17) | | | |
| V2.2 | GUUUGAGAGUAGUGUAAGAAAUUACACUACAAGUUCAAAUAA | | | 85 |
| | AAAUUUAUUCAAAUCCAUUUGCUACAUUGUGUAGAAUUUUUU | | | |
| | U (SEQ ID NO: 18) | | | |
| V2.3 | GUUUGAGAGUAGUGGAAACACUACAAGUUCAAAUAAAAAUUU | | | 79 |
| | AUUCAAAUCCAUUUGCUACAUUGUGUAGAAUUUUUUU | | | |
| | (SEQ IDNO: 19) | | | |
| V2.4 | GUUUGAGAGUAGUGGAAACACUACAAGUUCAAAUAAAAAUUU | | | 95 |
| | AUUCAAAUCCAUUUGCUACAUUGUGUAGAAUUUAAAGAUGCA | | | |
| | AAUCUUUUUUU (SEQ ID NO: 20) | | | |
| V2.5 | GUUUGAGAGUAGUGUAAGAAAUUACACUACAAGUUCAAAUAA | | | 101 |
| | AAAUUUAUUCAAAUCCAUUUGCUACAUUGUGUAGAAUUUAAA | | | |
| | GAUGCAAAUCUUUUUUU (SEQ ID NO: 21) | | | |
| Experimental | | | | |
| Name | crRNA Repeat | | tracrRNA anti-repeat | |
| V2.1 | GUUUGAGAGUAGUG (SEQ ID NO: 22) | | CACUACAAGUUCAAAU (SEQ ID | |
| | | | NO: 24) | |
| V2.2 | GUUUGAGAGUAGUGUAA (SEQ ID | | UUACACUACAAGUUCAAAU (SEQ | |
| | NO: 23) | | ID NO: 25) | |
| V2.3 | GUUUGAGAGUAGUG (SEQ ID NO: 22) | | CACUACAAGUUCAAAU (SEQ ID | |
| | | | NO: 24) | |
| V2.4 | GUUUGAGAGUAGUG (SEQ ID NO: 22) | | CACUACAAGUUCAAAU (SEQ ID | |
| | | | NO: 24) | |
| V2.5 | GUUUGAGAGUAGUGUAA (SEQ ID | | UUACACUACAAGUUCAAAU (SEQ | |
| | NO: 23) | | ID NO: 25) | |
| | | tracrRNA | | tracrRNA |
| Experimental | | Portion | tracrRNA | Portion |
| Name | tracrRNA Portion 1 | 1-partial | Portion 2 | 2-partial |
| V2.1 | AAAAAUUUAUUCA | Not listed | AAAGAUCUGG | AAAGAUCUGG |
| | AAUCCAUUUGCUA | | CAACAGAUCU | CAACAGA (SEQ |
| | CAUUGUGUAGAAU | | UUUUUU (SEQ | ID NO: 31) |
| | UU (SEQ ID NO: 26) | | ID NO: 29) | |
| V2.2 | AAAAAUUUAUUCA | AAAAAUUUAU | Not listed | Not listed |
| | AAUCCAUUUGCUA | UCAAAUCCAU | | |
| | CAUUGUGUAGAAU | UUGCUACAUU | | |
| | UUUUUU (SEQ ID | GUGUAGAA | | |
| | NO: 27) | (SEQ ID | | |
| | | NO: 28) | | |
| V2.3 | AAAAAUUUAUUCA | AAAAAUUUAU | Not listed | Not listed |
| | AAUCCAUUUGCUA | UCAAAUCCAU | | |
| | CAUUGUGUAGAAU | UUGCUACAUU | | |
| | UUUUUU (SEQ ID | GUGUAGAA | | |
| | NO: 27) | (SEQ ID | | |
| | | NO: 28) | | |
| V2.4 | AAAAAUUUAUUCA | Not listed | AAAGAUGCAA | AAAGAUGCAA |
| | AAUCCAUUUGCUA | | AUCUUUUUUU | AUC (SEQ ID |
| | CAUUGUGUAGAAU | | (SEQ ID | NO: 32) |
| | UU (SEQ ID NO: 26) | | NO: 30) | |
| V2.5 | AAAAAUUUAUUCA | Not listed | AAAGAUGCAA | AAAGAUGCAA |
| | AAUCCAUUUGCUA | | AUCUUUUUUU | AUC (SEQ ID |
| | CAUUGUGUAGAAU | | (SEQ ID | NO: 32) |
| | UU (SEQ ID NO: 26) | | NO: 30) | |

TABLE 4
Endogenic targets for testing activity
short-scaffold guide activity

| Gene | Site | Spacer |
|---|---|---|
| TRAC | s91 | GCUGUGGCCUGGAGCAACAAAU (SEQ ID NO: 33) |
| PDCD1 | s40 | AACACAUCGGAGAGCUUCGUGC (SEQ ID NO: 34) |
| B2M | S12 | GUAUGCCUGCCGUGUGAACCAU (SEQ ID NO: 35) |
| TRAC | S35 | GACCCUGCCGUGUACCAGCUGA (SEQ ID NO: 36) |

TABLE 5
Summary of the activity panel of short guides across
different endogenic targets in three cell types

| Cell Type | Gene | Site | V2 | V2.1 | V2.2 | V2.3 | V2.4 | V2.5 |
|---|---|---|---|---|---|---|---|---|
| HeLa | TRAC | s91 | 46.90 | 35.81 | 42.68 | 42.74 | 38.62 | 4.06 |
| HeLa | PDCD1 | s40 | 35.21 | 9.45 | 32.94 | 40.82 | 27.60 | 25.59 |
| U2OS | TRAC | S35 | 96.58 | N/A | 96.22 | 97.34 | N/A | N/A |
| U20S | B2M | S12 | 97.19 | N/A | 97.76 | 96.16 | N/A | N/A |
| Primary T | TRAC | S35 | 77.40 | N/A | 87.60 | 90.80 | N/A | N/A |
| cells | | | | | | | | |
| Primary T | B2M | S12 | 79.90 | N/A | 88.00 | 91.60 | N/A | N/A |
| cells | | | | | | | | |

TABLE 6
Summary of the sgRNAs used in the primary T cell assays

| sgRNA Name | Gene | Site | Spacer | Scaffold | sgRNA |
|---|---|---|---|---|---|
| OMNI-103 v2.2 | TRAC | S35 | SEQ ID | SEQ ID | SEQ ID |
| TRAC S35 | | | NO: 36 | NO: 18 | NO: 37 |
| OMNI-103 v2.3 | TRAC | S35 | SEQ ID | SEQ ID | SEQ ID |
| TRAC S35 | | | NO: 36 | NO: 19 | NO: 38 |
| OMNI-103 v2.2 | B2M | S12 | SEQ ID | SEQ ID | SEQ ID |
| B2M S12 | | | NO: 35 | NO: 18 | NO: 39 |
| OMNI-103 v2.3 | B2M | S12 | SEQ ID | SEQ ID | SEQ ID |
| B2M S12 | | | NO: 35 | NO: 19 | NO: 40 |

### REFERENCES

1. Ahmad and Allen (1992) “Antibody-mediated Specific Binding and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Basha et al. (2011) “Influence of Cationic Lipid Composition on Gene Silencing Properties of Lipid Nanoparticle Formulations of siRNA in Antigen-Presenting Cells”, Mol. Ther. 19 (12): 2186-200.
4. Behr (1994) “Gene transfer with synthetic cationic amphiphiles: Prospects for gene therapy”, Bioconjuage Chem 5:382-89.
5. Blaese et al. (1995) “Vectors in cancer therapy: how will they deliver”, Cancer Gene Ther. 2:291-97.
6. Blaese et al. (1995) “T lympocyte-directed gene therapy for ADA-SCID: initial trial results after 4 years”, Science 270 (5235): 475-80.
7. Briner et al. (2014) “Guide RNA functional modules direct Cas9 activity and orthognality”, Molecular Cell 56:333-39.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Canver et al., (2015) “BCL11A enhancer dissection by Cas9-mediated in situ saturating mutagenesis”, Nature Vol. 527, Pgs. 192-214.
11. Chang and Wilson (1987) “Modification of DNA ends can decrease end-joining relative to homologous recombination in mammalian cells”, Proc. Natl. Acad. Sci. USA 84:4959-4963.
12. Charlesworth et al. (2019) “Identification of preexisting adaptive immunity to Cas9 proteins in humans”, Nature Medicine, 25 (2), 249.
13. Chung et al. (2006) “Agrobacterium is not alone: gene transfer to plants by viruses and other bacteria”, Trends Plant Sci. 11 (1): 1-4.
14. Coelho et al. (2013) “Safety and efficacy of RNAi therapy for transthyretin amyloidosis” N. Engl. J. Med. 369, 819-829.
15. Crystal (1995) “Transfer of genes to humans: early lessons and obstacles to success”, Science 270 (5235): 404-10.
16. Dillon (1993) “Regulation gene expression in gene therapy” Trends in Biotechnology 11 (5): 167-173.
17. Dranoff et al. (1997) “A phase I study of vaccination with autologous, irradiated melanoma cells engineered to secrete human granulocyte macrophage colony stimulating factor”, Hum. Gene Ther. 8 (1): 111-23.
18. Dunbar et al. (1995) “Retrovirally marked CD34-enriched peripheral blood and bone marrow cells contribute to long-term engraftment after autologous transplantation”, Blood 85:3048-57.
19. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
20. Gao and Huang (1995) “Cationic liposome-mediated gene transfer” Gene Ther. 2 (10): 710-22.
21. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
22. Han et al. (1995) “Ligand-directed retro-viral targeting of human breast cancer cells”, Proc. Natl. Acad. Sci. USA 92 (21): 9747-51.
23. Humbert et al., (2019) “Therapeutically relevant engraftment of a CRISPR-Cas9-edited HSC-enriched population with HbF reactivation in nonhuman primates”, Sci. Trans. Med., Vol. 11, Pgs. 1-13.
24. Inaba et al. (1992) “Generation of large numbers of dendritic cells from mouse bone marrow cultures supplemented with granulocyte/macrophage colony-stimulating factor”, J Exp Med. 176 (6): 1693-702.
25. Jiang and Doudna (2017) “CRISPR-Cas9 Structures and Mechanisms”, Annual Review of Biophysics 46:505-29.
26. Jinek et al. (2012) “A programmable dual-RNA-guided DNA endonuclease in adaptive bacterial immunity”, Science 337 (6096): 816-21.
27. Johan et al. (1992) “GLVR1, a receptor for gibbon ape leukemia virus, is homologous to a phosphate permease of Neurospora crassa and is expressed at high levels in the brain and thymus”, J Virol 66 (3): 1635-40.
28. Judge et al. (2006) “Design of noninflammatory synthetic siRNA mediating potent gene silencing in vivo”, Mol Ther. 13 (3): 494-505.
29. Kohn et al. (1995) “Engraftment of gene-modified umbilical cord blood cells in neonates with adnosine deaminase deficiency”, Nature Medicine 1:1017-23.
30. Kremer and Perricaudet (1995) “Adenovirus and adeno-associated virus mediated gene transfer”, Br. Med. Bull. 51 (1): 31-44.
31. Macdiarmid et al. (2009) “Sequential treatment of drug-resistant tumors with targeted minicells containing siRNA or a cytotoxic drug”, Nat Biotehcnol. 27 (7): 643-51.
32. Malech et al. (1997) “Prolonged production of NADPH oxidase-corrected granulocyes after gene therapy of chronic granulomatous disease”, PNAS 94 (22): 12133-38.
33. Maxwell et al. (2018) “A detailed cell-free transcription-translation-based assay to decipher CRISPR protospacer adjacent motifs”, Methods 14348-57
34. Miller et al. (1991) “Construction and properties of retrovirus packaging cells based on gibbon ape leukemia virus”, J Virol. 65 (5): 2220-24.
35. Miller (1992) “Human gene therapy comes of age”, Nature 357:455-60.
36. Mir et al. (2019) “Type II-C CRISPR-Cas9 Biology, Mechanism and Application”, ACS Chem. Biol. 13 (2): 357-365.
37. Mitani and Caskey (1993) “Delivering therapeutic genes-matching approach and application”, Trends in Biotechnology 11 (5): 162-66.
38. Nabel and Felgner (1993) “Direct gene transfer for immunotherapy and immunization”, Trends in Biotechnology 11 (5): 211-15.
39. Nehls et al. (1996) “Two genetically separable steps in the differentiation of thymic epithelium” Science 272:886-889.
40. Nishimasu et al. “Crystal structure of Cas9 in complex with guide RNA and target DNA” (2014) Cell 156 (5): 935-49.
41. Nishimasu et al. (2015) “Crystal Structure of Staphylococcus aureus Cas9” Cell 162 (5): 1113-26.
42. Palermo et al. (2018) “Key role of the REC lobe during CRISPR-Cas9 activation by ‘sensing’, ‘regulating’, and ‘locking’ the catalytic HNH domain” Quarterly Reviews of Biophysics 51, e9, 1-11.
43. Remy et al. (1994) “Gene Transfer with a Series of Lipphilic DNA-Binding Molecules”, Bioconjugate Chem. 5 (6): 647-54.
44. Sentmanat et al. (2018) “A Survey of Validation Strategies for CRISPR-Cas9 Editing”, Scientific Reports 8:888, doi: 10.1038/s41598-018-19441-8.
45. Sommerfelt et al. (1990) “Localization of the receptor gene for type D simian retroviruses on human chromosome 19”, J. Virol. 64 (12): 6214-20.
46. Van Brunt (1988) “Molecular framing: transgenic animals as bioactors” Biotechnology 6:1149-54.
47. Vigne et al. (1995) “Third-generation adenovectors for gene therapy”, Restorative Neurology and Neuroscience 8 (1,2): 35-36.
48. Wagner et al. (2019) “High prevalence of Streptococcus pyogenes Cas9-reactive T cells within the adult human population” Nature Medicine, 25 (2), 242
49. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
50. Yu et al. (1994) “Progress towards gene therapy for HIV infection”, Gene Ther. 1 (1): 13-26.
51. Zetsche et al. (2015) “Cpf1 is a single RNA-guided endonuclease of a class 2 CRIPSR-Cas system” Cell 163 (3): 759-71.
52. Zuris et al. (2015) “Cationic lipid-mediated delivery of proteins enables efficient protein based genome editing in vitro and in vivo” Nat Biotechnol. 33 (1): 73-80.

What is claimed is:

1. A composition comprising a non-naturally occurring RNA molecule, or a polynucleotide molecule encoding the RNA molecule, the RNA molecule comprising a crRNA repeat sequence portion and a guide sequence portion, wherein the RNA molecule forms a complex with and targets an OMNI-103 nuclease to a DNA target site in the presence of a tracrRNA sequence, wherein the tracrRNA sequence is encoded by a tracrRNA portion of the RNA molecule or a tracrRNA portion of a second RNA molecule.

2. The composition of claim 1, wherein the crRNA repeat sequence portion is up to 17 nucleotides in length, preferably 14-17 nucleotides in length.

3. The composition of claim 1 or claim 2, wherein the crRNA repeat sequence portion has at least 60-70%, 71-80%, 81-90%, 91-95%, or 96-99% sequence identity to SEQ ID NOs: 22 or 23.

4. The composition of any one of claims 1-3, wherein the crRNA repeat sequence portion has at least 95% sequence identity to any one of SEQ ID NOs: 22 or 23.

5. The composition of any one of claims 1-4, wherein the crRNA repeat sequence is other than SEQ ID NO: 23.

6. The composition of any one of claims 1-5, wherein the RNA molecule comprising the crRNA repeat sequence portion and the guide sequence portion further comprises the tracrRNA portion.

7. The composition of claim 6, wherein the crRNA repeat sequence portion is covalently linked to the tracrRNA portion by a polynucleotide linker portion.

8. The composition of any one of claims 1-5, wherein the composition comprises a second RNA molecule comprising the tracrRNA portion.

9. The composition of any one of claims 1-8, wherein the OMNI-103 nuclease has at least 95% sequence identity to the amino acid sequence of SEQ ID NO: 1.

10. The composition of any one of claims 1-9, wherein the guide sequence portion is 17-30 nucleotides in length, preferably 22 nucleotides in length.

11. A composition comprising a non-naturally occurring RNA molecule, the RNA molecule comprising a tracrRNA portion, wherein the RNA molecule forms a complex with and targets an OMNI-103 nuclease to a DNA target site in the presence of a crRNA repeat sequence portion and a guide sequence portion, wherein the crRNA repeat sequence portion and the guide sequence portion are encoded by the RNA molecule or a second RNA molecule.

12. The composition of claim 11, wherein the tracrRNA portion is less than 85 nucleotides in length, preferably 84-80, 79-75, 74-70, 69-65, or 64-60 nucleotides in length.

13. The composition of claim 11 or 12, wherein the tracrRNA portion has at least 30-40%, 41-50%, 51-60%, 61-70%, 71-80%, 81-90%, 91-95%, or 96-99% sequence identity to the tracrRNA portion of any one of SEQ ID NOs: 17-21.

14. The composition of any one of claims 11-13, wherein the tracrRNA portion has at least 95% sequence identity to a tracrRNA portion of any one of SEQ ID NOs: 17-21.

15. The composition of any one of claims 11-14, wherein the tracrRNA portion is other than the tracr portion of SEQ ID NO: 4 or 5.

16. The composition of any one of claims 11-15, wherein the tracrRNA portion comprises a tracrRNA anti-repeat sequence portion that is up to 19 nucleotides in length, preferably 16-19 nucleotides in length.

17. The composition of any one of claims 11-16, wherein the tracrRNA portion comprises a tracrRNA anti-repeat sequence portion that has at least 60-70%, 71-80%, 81-90%, 91-95%, or 96-99% sequence identity to any one of SEQ ID NOs: 24 or 25.

18. The composition of any one of claims 11-17, wherein the tracrRNA portion comprises a tracrRNA anti-repeat sequence portion that has at least 95% sequence identity to any one of SEQ ID NOs: 24 or 25.

19. The composition of any one of claims 11-18, wherein the tracrRNA portion comprises a tracrRNA anti-repeat sequence portion having a sequence other than SEQ ID NO: 25.

20. The composition of any one of claims 11-19, wherein the RNA molecule comprises a tracrRNA portion and further comprises a crRNA repeat sequence portion and a guide sequence portion.

21. The composition of any one of claims 11-20, wherein the tracrRNA portion is covalently linked to the crRNA repeat sequence by a polynucleotide linker portion.

22. The composition of claim 21, wherein the polynucleotide linker portion is 4-10 nucleotides in length.

23. The composition of claim 22, wherein the polynucleotide linker has a sequence of GAAA.

24. The composition of any one of claims 11-19, wherein the composition further comprises a second RNA molecule comprising a crRNA repeat sequence portion and a guide sequence portion.

25. The composition of any one of claims 11-24, wherein the OMNI-103 nuclease is at least 95% sequence identity to the amino acid sequence of SEQ ID NO: 1.

26. The composition of any one of claims 11-25, wherein the guide sequence portion is 17-30 nucleotides in length, preferably 22 nucleotides in length.

27. A composition comprising a non-naturally occurring RNA molecule, or a polynucleotide molecule encoding the RNA molecule, the RNA molecule comprising an RNA scaffold portion, the RNA scaffold portion having the structure:
crRNA repeat sequence portion-tracrRNA portion;
wherein the RNA scaffold portion forms a complex with and targets an OMNI-103 CRISPR nuclease to a DNA target site having complimentarity to a guide sequence portion of the RNA molecule.

28. The composition of claim 27, wherein the OMNI-103 nuclease has at least 95% sequence identity to the amino acid sequence of SEQ ID NO: 1.

29. The composition of claim 27 or 28, wherein the RNA scaffold portion is 110-105, 104-100, 99-95, 94-90, 89-85, 84-80, 79-75, or 74-70 nucleotides in length.

30. The composition of any one of claims 27-29, wherein the RNA scaffold portion is 107, 101, 95, 85, or 79 nucleotides in length.

31. The composition of any one of claims 27-30, wherein the RNA scaffold portion has at least 30%, at least 40%, at least 50%, at least 60%, at least 70%, at least 80%, at least 85%, at least 90%, or at least 95% sequence identity to any one of SEQ ID NOs: 17-21.

32. The composition of any one of claims 27-31, wherein the crRNA repeat sequence portion is up to 17 nucleotides in length, preferably 14-17 nucleotides in length.

33. The composition any one of claims 27-32, wherein the crRNA repeat sequence portion has at least 60-70%, 71-80%, 81-90%, 91-95%, or 96-99% sequence identity to SEQ ID NOs:
22 or 23.

34. The composition of any one of claims 27-33, wherein the crRNA repeat sequence portion has at least 95% sequence identity to any one of SEQ ID NOs: 22 or 23.

35. The composition of any one of claims 27-34, wherein the crRNA repeat sequence is other than SEQ ID NO: 23.

36. The composition of any one of claims 27-35, wherein the tracrRNA portion is less than 85 nucleotides in length, preferably 84-80, 79-75, 74-70, 69-65, or 64-60 nucleotides in length.

37. The composition of any one of claims 27-36, wherein the tracrRNA portion has at least 30-40%, 41-50%, 51-60%, 61-70%, 71-80%, 81-90%, 91-95%, or 96-99% sequence identity to the tracrRNA portion of any one of SEQ ID NOs: 17-21.

38. The composition of any one of claims 27-37, wherein the tracrRNA portion has at least 95% sequence identity to the tracrRNA portions of any one of SEQ ID NOs: 17-21.

39. The composition of any one of claims 27-38, wherein the tracrRNA portion is other than the tracrRNA portion of SEQ ID NO: 4 or 5.

40. The composition of any one of claims 27-39, wherein the RNA scaffold portion further comprises a linker portion between the crRNA repeat sequence portion and the tracrRNA portion such that the RNA scaffold has the structure:
crRNA repeat sequence portion-linker portion-tracrRNA portion.

41. The composition of any one of claims 27-40, wherein the tracrRNA portion comprises a tracrRNA anti-repeat sequence portion, wherein the crRNA repeat sequence and the tracrRNA anti-repeat sequence portion are covalently linked by the linker portion.

42. The composition of claim 41, wherein the linker portion is a polynucleotide linker that is 4-10 nucleotides in length.

43. The composition of claim 42, the polynucleotide linker has a sequence of GAAA.

44. The composition of any one of claims 27-43, wherein the tracrRNA portion comprises a tracrRNA anti-repeat sequence portion that is up to 19 nucleotides in length, preferably 16-19 nucleotides in length.

45. The composition of any one of claims 27-44, wherein the tracrRNA portion comprises a tracrRNA anti-repeat sequence portion that has at least 60-70%, 71-80%, 81-90%, 91-95%, or 96-99% sequence identity to any one of SEQ ID NOs: 24 or 25.

46. The composition of any one of claims 27-45, wherein the tracrRNA portion comprises a tracrRNA anti-repeat sequence portion that has at least 95% sequence identity to any one of SEQ ID NOs: 24 or 25.

47. The composition of any one of claims 27-46, wherein the tracrRNA anti-repeat sequence is other than SEQ ID NO: 25.

48. The composition of any one of claims 27-47, wherein the tracrRNA portion comprises a first section of nucleotides linked to the tracrRNA anti-repeat portion, and the first section of nucleotides has at least 95% sequence identity to any one of SEQ ID NOs: 26-28.

49. The composition of any one of claims 27-48, wherein the tracrRNA portion comprises a second section of nucleotides linked to a first section of nucleotides, and the second section of nucleotides has at least 95% sequence identity to any one of SEQ ID NOs: 29-32.

50. The composition of any one of claims 27-49, wherein the RNA scaffold portion has at least 95% identity to the nucleotide sequence of any one of SEQ ID NOs: 17-21.

51. The composition of any one of claims 27-50, wherein the RNA scaffold portion has a predicted structure of any one of the V2, V2.1, V2.2, V2.3, V2.4, or V2.5 RNA scaffolds.

52. The composition of any one of claims 27-51, wherein the RNA scaffold portion has a sequence other than SEQ ID NO: 4 or 5.

53. The composition of any one of claims 27-52, wherein a guide sequence portion is covalently linked to the crRNA repeat sequence portion of the RNA molecule, forming a single-guide RNA molecule having a structure:
guide sequence portion-crRNA repeat sequence portion-tracrRNA portion.

54. The composition of any one of claims 27-53, wherein the guide sequence portion is 17-30 nucleotides, more preferably 20-23 nucleotides, more preferably 22 nucleotides in length.

55. The composition of any one of claims 1-54, further comprising an OMNI-103 CRISPR nuclease, wherein the OMNI-103 CRISPR nuclease has at least 95% identity to the amino acid sequence of SEQ ID NO: 1.

56. The composition of any one of claims 1-55, wherein the RNA molecule is formed by in vitro transcription (IVT) or solid-phase artificial oligonucleotide synthesis.

57. The composition of claim 56, wherein the RNA molecule comprises modified nucleotides.

58. The composition of any one of claims 1-57, wherein the RNA molecule comprises a sequence of any one of SEQ ID NOs: 17-21.

59. The compoistion of claim 58, wherein the RNA molecule comprises a sequence of SEQ ID NO: 18.

60. The compoistion of claim 58, wherein the RNA molecule comprises a sequence of SEQ ID NO: 19.

61. The composition of any one of claims 58-60, wherein the RNA molecule consists of a guide sequence portion and a sequence of any one of SEQ ID NOs: 17-21.

62. A polynucleotide molecule encoding the RNA molecule of any one of claims 1-61.

63. A method of modifying a nucleotide sequence at a DNA target site in a cell-free system or a genome of a cell comprising introducing into the system or cell the composition of any one of claims 1-61 and a CRISPR nuclease having at least 95% sequence identity to the amino acid sequence of SEQ ID NO: 1.

64. The method of claim 63, wherein the cell is a eukaryotic cell or a prokaryotic cell.

65. The method of claim 64, wherein the eukaryotic cell is a human cell or a plant cell.

66. A kit for modifying a nucleotide sequence at a DNA target site in a cell-free system or a genome of a cell comprising introducing into the system or cell the composition of any one of claims 1-61, a CRISPR nuclease having at least 95% sequence identity to the amino acid sequence of SEQ ID NO: 1, and instructions for delivering the RNA molecule and the CRISPR nuclease to the cell.


SEQUENCE LISTING (40 sequences)

SEQ ID NO: 1 - protein, 1348 aa, unidentified
  REGION 1..1348: note = OMNI-103 amino acid sequence
MSIKSDYFLGLDIGTDSIGWAVTDPEYHILRRKGKALWGIRLFDAANTAAERRTFRTSRRRIQRRRQRIRLLQELFAEEMVKLDPGFFQRLSDSAFWQEDKQEQQIYSLFTCENYTDVDYYREYPTIYHLRSALIQEKKEFDLRLLYLALHHLMKHRGHFLFNGSINNVTSFHTTFQTFADCLYDEFDIELECDSEDRFAEILKDKHARKTGKCSELEIICHIEKSNKQLKELFKLITGMKASLSVVFGDDELAEIEHNKISFSESSYDEVRLALEDEIQERTGILDIFHAVYSWAILADILEGGEYEGNSYLSVAKVSTYKKHGDDLRLLRTLVREYCPDHYKSFFSVSGKENYCAYAGTLKKNGKKQPIKRCSQEDFYKALKKLLNQMPTEQPEVKDIFIEIENGTFLPLQVSKDNGVIPYQVNKMELEKILQNAEEYLPFLKNIDEECGKTVSKKIIDLFEFRIPYYVGPLNTAKGENCWMVRKEAGRIYPWNFDEKVDRDQSAEKFIRRMTNQCTYLIHEDVVPKNSLLYSEFMVLNELNNVKIRSEKLPVELKQAIVLDLFKKQKQITGKKLLNYLNANGYDVKKEDLSGFDGNFKSSLSSYLTLKKVFGEELDKYSVQQMAEDIILWITLYGDDQKMLRRVIRKHYEQQLSEEQILSLSKLKFQGWGRLSRRLLSEMEGVDCETGECMTVMQGLRNTQNNLMQLLSQQFSFMELIEEENGNYYVDEITYDNLVKDMVISPSVKRAVWQTVQIVEEIKGVMGCQPKKIFVEMARSDEEKKRTVSRKDRLLEAYDAIKDEARQWQEELQKYSDGDFKAIKLYLYYTQMGQCMYTGRKIDLSQLNDATVWDRDHIYPQSKTKDDSLDNLVLVDRSVNAKKSDGMLSPEIQQRMRATWKYLKEKKLISEKKYERLTRVSPLTDEELAGFINRQLVETRQSSKAVATLLKRVYDEAEIVYVKAEAVSNFRRDNLDYIKVRDLNDYHHAKDAYQNIVVGNVFHEKFTSNPLRWLKNNPNTKYSLNQMFNFDLEKNGVVIWKRGKAGSIKCVEETLKRNDILFTRYAFCNKGGFFNQMLTAAPEDKTKAKGLVPIKKGMETWKYGGYTSVTPSHFMLVASKDKKGKEIRTIETVPLYRWKEFKENPDALLQYCREFYGLKEPKVLIPCIKKNARLVVNGFPMHLKGSTGKQLILQGAVQLCLNNENIKYLKKVTKYLEYNAQRRDKRTLLEVREVTGINKEENIQLYDVFVDKLSNTIYQYRPANPKDNLIKGREKFIELGLAEQCVVLGEVLHLFQCKPLTSDLTLIGGSPNTGTIKITKTISNCNVVKLLSQSIAGVKVREINLLII

SEQ ID NO: 2 - DNA, 4047 bp, unidentified
  misc_feature 1..4047: note = OMNI-103 DNA sequence
  source 1..4047: mol_type = other DNA
atgagcataaaaagtgattattttttaggacttgatattggtacggattctattggatgggcggtaaccgacccagaatatcacatattgagacgaaaaggtaaagcattatggggaataagattatttgatgcggccaatacagcggcagaacggcgaacatttaggacaagccgaagaaggattcagagaagacgacagagaattcggttattgcaagaattatttgcagaagaaatggtaaaattagacccaggattttttcagaggttgtcagacagcgcattttggcaggaggataagcaagagcagcaaatttattcactttttacttgtgaaaattatacagatgttgattattacagagaatatcctactatttatcatttgagaagtgcattgattcaggaaaagaaggaatttgatcttcgtcttctatatcttgctcttcaccatttgatgaagcacaggggacatttcctgtttaatgggagtattaataatgtgacgtcatttcatacgacgtttcagacgtttgcagattgtctttatgatgagtttgatatagaactggaatgtgattccgaagatagatttgcagaaattttaaaggataaacatgccagaaaaacaggaaaatgttctgaattagagataatctgtcatatagaaaaatcaaataagcagctaaaagaactttttaaattaattacaggaatgaaagctagtttgagtgttgtgtttggtgatgatgagttagcggaaatagaacataataagattagtttttcagagagtagttatgatgaagtacgtcttgcattggaggatgagattcaggagaggactggtatactggatatctttcatgcagtttatagttgggcgattctcgcggatattttagaaggcggagaatatgaggggaattcttatctaagcgttgcgaaggtaagcacttataaaaagcatggtgatgatttgcggttgctaagaacactggttcgggaatattgtcctgatcattacaaatctttcttttccgtatcagggaaggagaattattgtgcatatgcgggtactttaaaaaagaatggaaaaaaacagccgattaaacgttgcagccaggaagatttttataaagcgttaaagaaattgctgaatcagatgccgacagaacaaccagaagtgaaagacatcttcatcgaaattgaaaatggtacttttttgccgttgcaggtaagtaaggataatggagtgataccttatcaggtaaataagatggaattagaaaaaatcctgcagaatgcagaggaatatttgccatttctaaaaaatatagatgaagaatgtggaaaaacggttagtaagaagattatagatctctttgagtttagaataccatattatgtagggccgcttaataccgctaaaggagaaaactgttggatggtcagaaaagaagcggggagaatatatccgtggaattttgatgaaaaggtagacagagatcaatcagcagaaaaatttatccgtagaatgacgaatcagtgtacatatttaatacatgaggatgttgtacctaaaaattctttgctttattcggagtttatggtgcttaatgaattaaataatgtaaagatccggtctgagaagctgccggtggagttaaaacaggcaatagtattggatttgtttaagaaacaaaagcagataacaggaaaaaaacttcttaattacttgaatgcaaatggatatgatgtaaaaaaagaagatttgtcagggtttgacggaaactttaaatcatctctgtcatcatatcttactttgaaaaaagtatttggtgaagaattagataaatatagtgtgcagcagatggcagaggatattatcttgtggatcactctgtatggagatgatcagaagatgttgcgcagggtaattcgaaaacattatgaacagcaattgagtgaagaacagattctttccttatcgaaattgaaattccaaggctggggaagattatccagacgacttttgagtgaaatggaaggcgttgattgtgagactggtgagtgtatgacggtcatgcaaggacttcgtaatactcagaataatctgatgcagcttctaagtcagcagttttcatttatggaattgattgaggaagaaaatgggaattattatgtagatgagattacatacgataatcttgtgaaagatatggttatatctccgtcagtgaagagagcagtctggcagacagttcagattgtggaggagattaagggggtaatgggctgtcagcctaagaagatatttgtcgagatggcgcgaagcgatgaagagaaaaagcgtactgtatctaggaaagacaggttattagaagcatatgatgcgatcaaggatgaggctcgtcaatggcaggaagagttgcaaaagtattcagatggtgattttaaggctattaaactttatctgtattatacgcagatggggcaatgtatgtatactggaagaaagatagatctgtcacaattaaatgatgcgacggtatgggacagagatcatatatatccacagtccaaaacaaaagatgatagtctggataatctggtattggtagaccggagcgtgaacgctaagaaaagtgatgggatgctatcacctgagattcagcagagaatgcgggctacttggaaatacttaaaagagaaaaagttgatttcagagaagaaatatgagcgtttgactagggtctcaccacttacagatgaggaattggcaggttttattaatcgacagttagttgaaacacgtcagtcttcgaaagcagtagcaacacttttgaaacgagtatatgatgaagcggagattgtctatgtaaaagcggaagctgtttcaaattttagaagagataatttggattatattaaggtgcgtgatctgaatgattatcatcatgctaaagatgcatatcagaatattgtagtggggaatgtttttcatgagaaatttaccagcaatccgcttcgttggctgaaaaacaatcctaatacgaaatatagtttaaatcagatgtttaactttgatttagagaaaaatggggtggtaatatggaaaagggggaaggctggaagtattaaatgtgttgaagaaacattgaaaagaaatgatattctttttacacgatatgctttttgtaataaaggtggtttttttaaccagatgttaacggcagctccagaagataaaacgaaagcaaagggacttgtaccaataaaaaaaggtatggaaacatggaaatacgggggatatacatcagtaactccgtcacattttatgttggttgcttcgaaagataagaaaggaaaggagataagaacgattgagacagttccgttgtataggtggaaagagttcaaagaaaatccagatgcattactccaatattgtagagagttctatggtttgaaagagcccaaggtgttgataccatgcatcaagaagaatgccagattagtcgttaatggatttccaatgcatttgaaagggagtacaggaaaacaattgattttgcagggagcggttcaattatgtctgaataatgaaaatataaagtatttgaaaaaagtcacaaaatatttggaatataatgcacagagaagagataaaagaacactgctggaagtaagagaggttacaggaattaacaaagaggaaaatatacagttatatgatgtgtttgttgataaattgagtaacacaatatatcagtatcgtccggccaatccaaaggacaatcttataaaaggaagagagaagtttatagaattagggttggcagaacaatgtgttgtgttaggtgaagtattgcatttgttccaatgtaaaccactcacgtctgatttgactttgattggaggttcaccgaatacagggacaataaaaataacaaagacaattagtaattgtaatgttgtaaagttgttaagccaatctattgcaggtgttaaagtgagagaaattaatttgttaataatatga

SEQ ID NO: 3 - DNA, 4041 bp, synthetic construct
  misc_feature 1..4041: note = OMNI-103 Human Optimized DNA sequence
  source 1..4041: mol_type = other DNA
tctatcaagagcgactacttcctgggcctcgacatcggcacagattctatcggatgggccgtgacagaccccgagtaccacatcctgagaagaaagggcaaagccctgtggggcatcagactgttcgatgccgccaatactgccgccgagagaagaaccttccggaccagcagaagaagaatccagcggcggagacagcggatcagactgctgcaagagctgttcgccgaggaaatggttaagctggaccccggcttcttccagagactgagcgatagcgccttctggcaagaggacaagcaagagcagcagatctacagcctgtttacctgcgagaactacaccgacgtggactactacagagagtaccccaccatctaccacctgagaagcgccctgatccaagagaaaaaagagttcgacctgcggctgctgtatctggccctgcaccatctgatgaagcaccggggccactttctgttcaacggcagcatcaacaacgtgaccagcttccacaccaccttccagaccttcgccgactgcctgtacgacgagttcgacatcgagctggaatgcgacagcgaggacagattcgccgagatcctgaaggataagcacgccagaaagaccggcaagtgctctgagctggaaatcatctgccacatcgagaagtccaacaagcagctgaaagaactgttcaagctgatcaccggcatgaaggccagcctgagcgtggtgtttggagatgatgagctggccgagatcgagcacaacaagatcagcttcagcgagagcagctacgacgaagtgcggctggccctggaagatgagattcaagagagaaccggcatcctggacatcttccacgccgtgtattcttgggccatcctggccgatattctggaaggcggcgagtacgagggcaacagctatctgtctgtggccaaggtgtccacctacaagaagcacggcgacgacctgagactgctgagaacactcgtgcgcgagtactgccccgaccactacaagagctttttcagcgtgtccggcaaagagaactactgcgcctacgccggcacactgaagaagaacggcaagaagcagcccatcaagcggtgcagccaagaggacttctacaaggccctgaagaaactgctgaaccagatgcctaccgagcagcccgaagtgaaggatatcttcatcgagattgagaacggcaccttcctgcctctgcaagtgtccaaggacaacggcgtgatcccctaccaagtgaacaagatggaactcgagaagatcctgcagaacgccgaagagtacctgcctttcctgaagaacatcgacgaggaatgcggcaagaccgtgtccaagaagatcatcgacctgttcgagttcagaatcccctactacgtgggccctctgaataccgccaagggcgagaattgctggatggttcgaaaagaggccggcagaatctacccctggaacttcgatgagaaggtggacagagatcagagcgccgagaagttcatcagacggatgaccaaccagtgcacctacctgatccacgaggacgtggtgcctaagaacagcctgctgtactccgagttcatggtgctgaacgagctgaacaatgtgaagattcggagcgagaagctgcccgtggaactgaagcaggccatcgtgctggacctgtttaagaagcagaagcagatcacagggaagaagctgctcaactacctgaacgccaacggctacgacgtgaagaaagaggacctgagcggcttcgacggcaacttcaagtccagcctgtccagctacctgactctgaagaaggtgttcggagaggaactggacaagtacagcgtgcagcagatggccgaggacatcatcctgtggatcaccctgtatggcgacgatcagaaaatgctgcggagagtgatccggaagcactacgagcagcagctgtctgaggaacagatcctgagcctgagcaagctgaagttccaaggctggggcagactgtctagacggctgctctctgaaatggaaggcgtggactgtgaaaccggcgagtgcatgacagtgatgcagggcctgagaaacacccagaacaacctgatgcagctgctgagccagcagttcagcttcatggaactgatcgaggaagagaacgggaactactacgtcgacgagatcacctacgacaacctggtcaaggacatggtcatcagccctagcgtgaaaagggccgtgtggcagacagtgcagatcgtggaagaaatcaagggcgtgatgggatgccagcctaagaaaatcttcgtggaaatggcccgcagcgacgaagagaagaaacggaccgtgtctcggaaggatcggctgctggaagcctacgacgccatcaaggatgaggcccggcaatggcaagaagaactgcagaaatactccgacggcgatttcaaggccatcaagctgtacctgtactacacccagatgggccagtgcatgtacaccggcagaaaaatcgatctgtcccagctgaacgacgccaccgtgtgggatagagatcacatctaccctcagagcaagaccaaggacgacagcctggacaatctggtgctggtggatagatccgtgaatgccaagaaaagcgacggcatgctgagccccgagatccagcagagaatgagagccacctggaagtacctgaaagaaaagaagctcatcagcgagaagaagtacgagcggctgaccagagtgtcccctctgacagatgaagaactggccggcttcatcaaccggcagctggtggaaacaagacagagcagcaaagccgtggccacactgctgaagagggtgtacgatgaggccgagattgtgtatgtgaaggccgaggccgtgtctaacttccggcgggataacctggactacatcaaagtgcgggacctgaacgactaccaccacgccaaggacgcctaccagaacatcgtcgtgggcaacgtgttccacgagaagtttaccagcaatcccctgcggtggctgaaaaacaaccccaacaccaagtactccctcaaccagatgttcaacttcgacctggaaaagaacggcgtggtcatctggaagagaggcaaggccggctccattaagtgtgtggaagagacactgaagcggaacgacatcctgttcaccagatacgctttctgcaacaaaggcggcttctttaatcagatgctgaccgccgctccagaggataagacaaaggccaaaggcctggtgcctatcaagaaaggcatggaaacctggaaatacggcggctacaccagcgtgacccctagccactttatgctggtggccagcaaggacaagaagggaaaagagatccggaccatcgagacagtgcccctgtaccggtggaaagagttcaaagagaatcccgacgctctgctccagtactgcagagagttctacggcctgaaagagcccaaggttctgatcccttgcatcaagaagaatgcccggctggtcgtgaacggcttccctatgcacctgaagggcagcaccggaaaacagctgattctgcagggtgccgtgcagctgtgcctgaacaacgagaacatcaagtacctcaagaaagtgacgaagtacctcgagtacaacgcccagcggagagacaagagaaccctgctcgaagttcgggaagtgaccggaatcaacaaagaggaaaacatccagctgtacgatgtgttcgtggacaagctgagcaacacaatctaccagtacagacccgccaatcctaaggacaacctcatcaagggccgcgagaaattcatcgagcttggcctggctgagcagtgcgtggtgctgggagaagtgctgcatctgttccagtgcaagcccctgaccagcgatctgacactgatcggcggaagccctaacaccggcaccatcaagatcaccaagaccatcagcaactgcaacgtggtcaagctgctgtcccagtctatcgccggcgtgaaagtccgcgagatcaacctgctgatcatc

SEQ ID NO: 4 - RNA, 107 nt, synthetic construct
  misc_feature 1..107: note = OMNI-103 sgRNA V1 scaffold
  source 1..107: mol_type = other RNA
gtttgagagtagtgtaagaaattacactacaagttcaaataaaaatttattcaaatccttttgctacattgtgtagaatttaaagatctggcaacagatcttttttt

SEQ ID NO: 5 - RNA, 107 nt, synthetic construct
  misc_feature 1..107: note = OMNI-103 sgRNA V2 scaffold
  source 1..107: mol_type = other RNA
gtttgagagtagtgtaagaaattacactacaagttcaaataaaaatttattcaaatccatttgctacattgtgtagaatttaaagatctggcaacagatcttttttt

SEQ ID NO: 6 - DNA, 6373 bp, synthetic construct
  misc_feature 1..6373: note = pET9a OMNI-103
  source 1..6373: mol_type = other DNA
taatacgactcactatagggagaccacaacggtttccctctagagagacaataaccctgataatgcttcaataatattgaaaaaggaagagtatgcctaagaagaagagaaaggtgggtacctctatcaagagcgactacttcctgggcctcgacatcggcacagattctatcggatgggccgtgacagaccccgagtaccacatcctgagaagaaagggcaaagccctgtggggcatcagactgttcgatgccgccaatactgccgccgagagaagaaccttccggaccagcagaagaagaatccagcggcggagacagcggatcagactgctgcaagagctgttcgccgaggaaatggttaagctggaccccggcttcttccagagactgagcgatagcgccttctggcaagaggacaagcaagagcagcagatctacagcctgtttacctgcgagaactacaccgacgtggactactacagagagtaccccaccatctaccacctgagaagcgccctgatccaagagaaaaaagagttcgacctgcggctgctgtatctggccctgcaccatctgatgaagcaccggggccactttctgttcaacggcagcatcaacaacgtgaccagcttccacaccaccttccagaccttcgccgactgcctgtacgacgagttcgacatcgagctggaatgcgacagcgaggacagattcgccgagatcctgaaggataagcacgccagaaagaccggcaagtgctctgagctggaaatcatctgccacatcgagaagtccaacaagcagctgaaagaactgttcaagctgatcaccggcatgaaggccagcctgagcgtggtgtttggagatgatgagctggccgagatcgagcacaacaagatcagcttcagcgagagcagctacgacgaagtgcggctggccctggaagatgagattcaagagagaaccggcatcctggacatcttccacgccgtgtattcttgggccatcctggccgatattctggaaggcggcgagtacgagggcaacagctatctgtctgtggccaaggtgtccacctacaagaagcacggcgacgacctgagactgctgagaacactcgtgcgcgagtactgccccgaccactacaagagctttttcagcgtgtccggcaaagagaactactgcgcctacgccggcacactgaagaagaacggcaagaagcagcccatcaagcggtgcagccaagaggacttctacaaggccctgaagaaactgctgaaccagatgcctaccgagcagcccgaagtgaaggatatcttcatcgagattgagaacggcaccttcctgcctctgcaagtgtccaaggacaacggcgtgatcccctaccaagtgaacaagatggaactcgagaagatcctgcagaacgccgaagagtacctgcctttcctgaagaacatcgacgaggaatgcggcaagaccgtgtccaagaagatcatcgacctgttcgagttcagaatcccctactacgtgggccctctgaataccgccaagggcgagaattgctggatggttcgaaaagaggccggcagaatctacccctggaacttcgatgagaaggtggacagagatcagagcgccgagaagttcatcagacggatgaccaaccagtgcacctacctgatccacgaggacgtggtgcctaagaacagcctgctgtactccgagttcatggtgctgaacgagctgaacaatgtgaagattcggagcgagaagctgcccgtggaactgaagcaggccatcgtgctggacctgtttaagaagcagaagcagatcacagggaagaagctgctcaactacctgaacgccaacggctacgacgtgaagaaagaggacctgagcggcttcgacggcaacttcaagtccagcctgtccagctacctgactctgaagaaggtgttcggagaggaactggacaagtacagcgtgcagcagatggccgaggacatcatcctgtggatcaccctgtatggcgacgatcagaaaatgctgcggagagtgatccggaagcactacgagcagcagctgtctgaggaacagatcctgagcctgagcaagctgaagttccaaggctggggcagactgtctagacggctgctctctgaaatggaaggcgtggactgtgaaaccggcgagtgcatgacagtgatgcagggcctgagaaacacccagaacaacctgatgcagctgctgagccagcagttcagcttcatggaactgatcgaggaagagaacgggaactactacgtcgacgagatcacctacgacaacctggtcaaggacatggtcatcagccctagcgtgaaaagggccgtgtggcagacagtgcagatcgtggaagaaatcaagggcgtgatgggatgccagcctaagaaaatcttcgtggaaatggcccgcagcgacgaagagaagaaacggaccgtgtctcggaaggatcggctgctggaagcctacgacgccatcaaggatgaggcccggcaatggcaagaagaactgcagaaatactccgacggcgatttcaaggccatcaagctgtacctgtactacacccagatgggccagtgcatgtacaccggcagaaaaatcgatctgtcccagctgaacgacgccaccgtgtgggatagagatcacatctaccctcagagcaagaccaaggacgacagcctggacaatctggtgctggtggatagatccgtgaatgccaagaaaagcgacggcatgctgagccccgagatccagcagagaatgagagccacctggaagtacctgaaagaaaagaagctcatcagcgagaagaagtacgagcggctgaccagagtgtcccctctgacagatgaagaactggccggcttcatcaaccggcagctggtggaaacaagacagagcagcaaagccgtggccacactgctgaagagggtgtacgatgaggccgagattgtgtatgtgaaggccgaggccgtgtctaacttccggcgggataacctggactacatcaaagtgcgggacctgaacgactaccaccacgccaaggacgcctaccagaacatcgtcgtgggcaacgtgttccacgagaagtttaccagcaatcccctgcggtggctgaaaaacaaccccaacaccaagtactccctcaaccagatgttcaacttcgacctggaaaagaacggcgtggtcatctggaagagaggcaaggccggctccattaagtgtgtggaagagacactgaagcggaacgacatcctgttcaccagatacgctttctgcaacaaaggcggcttctttaatcagatgctgaccgccgctccagaggataagacaaaggccaaaggcctggtgcctatcaagaaaggcatggaaacctggaaatacggcggctacaccagcgtgacccctagccactttatgctggtggccagcaaggacaagaagggaaaagagatccggaccatcgagacagtgcccctgtaccggtggaaagagttcaaagagaatcccgacgctctgctccagtactgcagagagttctacggcctgaaagagcccaaggttctgatcccttgcatcaagaagaatgcccggctggtcgtgaacggcttccctatgcacctgaagggcagcaccggaaaacagctgattctgcagggtgccgtgcagctgtgcctgaacaacgagaacatcaagtacctcaagaaagtgacgaagtacctcgagtacaacgcccagcggagagacaagagaaccctgctcgaagttcgggaagtgaccggaatcaacaaagaggaaaacatccagctgtacgatgtgttcgtggacaagctgagcaacacaatctaccagtacagacccgccaatcctaaggacaacctcatcaagggccgcgagaaattcatcgagcttggcctggctgagcagtgcgtggtgctgggagaagtgctgcatctgttccagtgcaagcccctgaccagcgatctgacactgatcggcggaagccctaacaccggcaccatcaagatcaccaagaccatcagcaactgcaacgtggtcaagctgctgtcccagtctatcgccggcgtgaaagtccgcgagatcaacctgctgatcatcggatcctacccatacgatgttccagattacgcggccgctccaaaaaagaaaagaaaagttgcggctagccatcatcaccatcaccatcatcattaaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcgatcccgcgaaat

SEQ ID NO: 7 - DNA, 2783 bp, synthetic construct
  misc_feature 1..2783: note = pShuttle Guide OMNI-103 V2
  source 1..2783: mol_type = other DNA
tacacggtgcctgactgcgttagcaatttaactgtgataaactaccgcattaaagcttatcgatgataagctgtcaacacatttccccgaaaagtgccacctgacgtcctcgagtcccgcataatcgaaatgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgctaatacgactcactataggaagagcagagccttggtctcgtttgagagtagtgtaagaaattacactacaagttcaaataaaaatttattcaaatccatttgctacattgtgtagaatttaaagatctggcaacagatctttttttgaattctctagcataaccccttggggcctctaaacgggtcttgaggggttttttgacctaggctaggggatatattccgggtaccccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttca

SEQ ID NO: 8 - DNA, 10286 bp, synthetic construct
  misc_feature 1..10286: note = pmOMNI OMNI-103
  source 1..10286: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgcctaagaagaagagaaaggtgggtacctctatcaagagcgactacttcctgggcctcgacatcggcacagattctatcggatgggccgtgacagaccccgagtaccacatcctgagaagaaagggcaaagccctgtggggcatcagactgttcgatgccgccaatactgccgccgagagaagaaccttccggaccagcagaagaagaatccagcggcggagacagcggatcagactgctgcaagagctgttcgccgaggaaatggttaagctggaccccggcttcttccagagactgagcgatagcgccttctggcaagaggacaagcaagagcagcagatctacagcctgtttacctgcgagaactacaccgacgtggactactacagagagtaccccaccatctaccacctgagaagcgccctgatccaagagaaaaaagagttcgacctgcggctgctgtatctggccctgcaccatctgatgaagcaccggggccactttctgttcaacggcagcatcaacaacgtgaccagcttccacaccaccttccagaccttcgccgactgcctgtacgacgagttcgacatcgagctggaatgcgacagcgaggacagattcgccgagatcctgaaggataagcacgccagaaagaccggcaagtgctctgagctggaaatcatctgccacatcgagaagtccaacaagcagctgaaagaactgttcaagctgatcaccggcatgaaggccagcctgagcgtggtgtttggagatgatgagctggccgagatcgagcacaacaagatcagcttcagcgagagcagctacgacgaagtgcggctggccctggaagatgagattcaagagagaaccggcatcctggacatcttccacgccgtgtattcttgggccatcctggccgatattctggaaggcggcgagtacgagggcaacagctatctgtctgtggccaaggtgtccacctacaagaagcacggcgacgacctgagactgctgagaacactcgtgcgcgagtactgccccgaccactacaagagctttttcagcgtgtccggcaaagagaactactgcgcctacgccggcacactgaagaagaacggcaagaagcagcccatcaagcggtgcagccaagaggacttctacaaggccctgaagaaactgctgaaccagatgcctaccgagcagcccgaagtgaaggatatcttcatcgagattgagaacggcaccttcctgcctctgcaagtgtccaaggacaacggcgtgatcccctaccaagtgaacaagatggaactcgagaagatcctgcagaacgccgaagagtacctgcctttcctgaagaacatcgacgaggaatgcggcaagaccgtgtccaagaagatcatcgacctgttcgagttcagaatcccctactacgtgggccctctgaataccgccaagggcgagaattgctggatggttcgaaaagaggccggcagaatctacccctggaacttcgatgagaaggtggacagagatcagagcgccgagaagttcatcagacggatgaccaaccagtgcacctacctgatccacgaggacgtggtgcctaagaacagcctgctgtactccgagttcatggtgctgaacgagctgaacaatgtgaagattcggagcgagaagctgcccgtggaactgaagcaggccatcgtgctggacctgtttaagaagcagaagcagatcacagggaagaagctgctcaactacctgaacgccaacggctacgacgtgaagaaagaggacctgagcggcttcgacggcaacttcaagtccagcctgtccagctacctgactctgaagaaggtgttcggagaggaactggacaagtacagcgtgcagcagatggccgaggacatcatcctgtggatcaccctgtatggcgacgatcagaaaatgctgcggagagtgatccggaagcactacgagcagcagctgtctgaggaacagatcctgagcctgagcaagctgaagttccaaggctggggcagactgtctagacggctgctctctgaaatggaaggcgtggactgtgaaaccggcgagtgcatgacagtgatgcagggcctgagaaacacccagaacaacctgatgcagctgctgagccagcagttcagcttcatggaactgatcgaggaagagaacgggaactactacgtcgacgagatcacctacgacaacctggtcaaggacatggtcatcagccctagcgtgaaaagggccgtgtggcagacagtgcagatcgtggaagaaatcaagggcgtgatgggatgccagcctaagaaaatcttcgtggaaatggcccgcagcgacgaagagaagaaacggaccgtgtctcggaaggatcggctgctggaagcctacgacgccatcaaggatgaggcccggcaatggcaagaagaactgcagaaatactccgacggcgatttcaaggccatcaagctgtacctgtactacacccagatgggccagtgcatgtacaccggcagaaaaatcgatctgtcccagctgaacgacgccaccgtgtgggatagagatcacatctaccctcagagcaagaccaaggacgacagcctggacaatctggtgctggtggatagatccgtgaatgccaagaaaagcgacggcatgctgagccccgagatccagcagagaatgagagccacctggaagtacctgaaagaaaagaagctcatcagcgagaagaagtacgagcggctgaccagagtgtcccctctgacagatgaagaactggccggcttcatcaaccggcagctggtggaaacaagacagagcagcaaagccgtggccacactgctgaagagggtgtacgatgaggccgagattgtgtatgtgaaggccgaggccgtgtctaacttccggcgggataacctggactacatcaaagtgcgggacctgaacgactaccaccacgccaaggacgcctaccagaacatcgtcgtgggcaacgtgttccacgagaagtttaccagcaatcccctgcggtggctgaaaaacaaccccaacaccaagtactccctcaaccagatgttcaacttcgacctggaaaagaacggcgtggtcatctggaagagaggcaaggccggctccattaagtgtgtggaagagacactgaagcggaacgacatcctgttcaccagatacgctttctgcaacaaaggcggcttctttaatcagatgctgaccgccgctccagaggataagacaaaggccaaaggcctggtgcctatcaagaaaggcatggaaacctggaaatacggcggctacaccagcgtgacccctagccactttatgctggtggccagcaaggacaagaagggaaaagagatccggaccatcgagacagtgcccctgtaccggtggaaagagttcaaagagaatcccgacgctctgctccagtactgcagagagttctacggcctgaaagagcccaaggttctgatcccttgcatcaagaagaatgcccggctggtcgtgaacggcttccctatgcacctgaagggcagcaccggaaaacagctgattctgcagggtgccgtgcagctgtgcctgaacaacgagaacatcaagtacctcaagaaagtgacgaagtacctcgagtacaacgcccagcggagagacaagagaaccctgctcgaagttcgggaagtgaccggaatcaacaaagaggaaaacatccagctgtacgatgtgttcgtggacaagctgagcaacacaatctaccagtacagacccgccaatcctaaggacaacctcatcaagggccgcgagaaattcatcgagcttggcctggctgagcagtgcgtggtgctgggagaagtgctgcatctgttccagtgcaagcccctgaccagcgatctgacactgatcggcggaagccctaacaccggcaccatcaagatcaccaagaccatcagcaactgcaacgtggtcaagctgctgtcccagtctatcgccggcgtgaaagtccgcgagatcaacctgctgatcatcggatcctacccatacgatgttccagattacgcggccgctccaaaaaagaaaagaaaagttgaattcggcggcagcggcgccaccaacttcagcctgctgaagcaggccggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - protein, 9 aa, synthetic construct
  REGION 1..9: note = HA Tag amino acid sequence
YPYDVPDYA

SEQ ID NO: 10 - protein, 7 aa, synthetic construct
  REGION 1..7: note = SV40 NLS amino acid sequence
PKKKRKV

SEQ ID NO: 11 - protein, 19 aa, synthetic construct
  REGION 1..19: note = P2A amino acid sequence
ATNFSLLKQAGDVEENPGP

SEQ ID NO: 12 - protein, 236 aa, synthetic construct
  REGION 1..236: note = mCherry amino acid sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 13 - DNA, 27 bp, synthetic construct
  misc_feature 1..27: note = HA Tag DNA sequence
  source 1..27: mol_type = other DNA
tacccatacgatgttccagattacgct

SEQ ID NO: 14 - DNA, 21 bp, synthetic construct
  misc_feature 1..21: note = SV40 NLS DNA sequence
  source 1..21: mol_type = other DNA
ccaaaaaagaaaagaaaagtt

SEQ ID NO: 15 - DNA, 57 bp, synthetic construct
  misc_feature 1..57: note = P2A DNA sequence
  source 1..57: mol_type = other DNA
gccaccaacttcagcctgctgaagcaggccggcgacgtggaggagaaccccggcccc

SEQ ID NO: 16 - DNA, 711 bp, synthetic construct
  misc_feature 1..711: note = mCherry DNA sequence
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 17 - RNA, 101 nt, synthetic construct
  misc_feature 1..101: note = V2.1 scaffold
  source 1..101: mol_type = other RNA
gtttgagagtagtggaaacactacaagttcaaataaaaatttattcaaatccatttgctacattgtgtagaatttaaagatctggcaacagatcttttttt

SEQ ID NO: 18 - RNA, 85 nt, synthetic construct
  misc_feature 1..85: note = V2.2 scaffold
  source 1..85: mol_type = other RNA
gtttgagagtagtgtaagaaattacactacaagttcaaataaaaatttattcaaatccatttgctacattgtgtagaattttttt

SEQ ID NO: 19 - RNA, 79 nt, synthetic construct
  misc_feature 1..79: note = V2.3 scaffold
  source 1..79: mol_type = other RNA
gtttgagagtagtggaaacactacaagttcaaataaaaatttattcaaatccatttgctacattgtgtagaattttttt

SEQ ID NO: 20 - RNA, 95 nt, synthetic construct
  misc_feature 1..95: note = V2.4 scaffold
  source 1..95: mol_type = other RNA
gtttgagagtagtggaaacactacaagttcaaataaaaatttattcaaatccatttgctacattgtgtagaatttaaagatgcaaatcttttttt

SEQ ID NO: 21 - RNA, 101 nt, synthetic construct
  misc_feature 1..101: note = V2.5 scaffold
  source 1..101: mol_type = other RNA
gtttgagagtagtgtaagaaattacactacaagttcaaataaaaatttattcaaatccatttgctacattgtgtagaatttaaagatgcaaatcttttttt

SEQ ID NO: 22 - RNA, 14 nt, synthetic construct
  misc_feature 1..14: note = crRNA Repeat sequence A
  source 1..14: mol_type = other RNA
gtttgagagtagtg

SEQ ID NO: 23 - RNA, 17 nt, synthetic construct
  misc_feature 1..17: note = crRNA Repeat sequence B
  source 1..17: mol_type = other RNA
gtttgagagtagtgtaa

SEQ ID NO: 24 - RNA, 16 nt, synthetic construct
  misc_feature 1..16: note = tracrRNA antirepeat sequence A
  source 1..16: mol_type = other RNA
cactacaagttcaaat

SEQ ID NO: 25 - RNA, 19 nt, synthetic construct
  misc_feature 1..19: note = tracrRNA antirepeat sequence B
  source 1..19: mol_type = other RNA
ttacactacaagttcaaat

SEQ ID NO: 26 - RNA, 41 nt, synthetic construct
  misc_feature 1..41: note = tracrRNA Portion 1 sequence A
  source 1..41: mol_type = other RNA
aaaaatttattcaaatccatttgctacattgtgtagaattt

SEQ ID NO: 27 - RNA, 45 nt, synthetic construct
  misc_feature 1..45: note = tracrRNA Portion 1 sequence B
  source 1..45: mol_type = other RNA
aaaaatttattcaaatccatttgctacattgtgtagaattttttt

SEQ ID NO: 28 - RNA, 38 nt, synthetic construct
  misc_feature 1..38: note = tracrRNA Portion 1 - partial sequence
  source 1..38: mol_type = other RNA
aaaaatttattcaaatccatttgctacattgtgtagaa

SEQ ID NO: 29 - RNA, 26 nt, synthetic construct
  misc_feature 1..26: note = tracrRNA Portion 2 sequence A
  source 1..26: mol_type = other RNA
aaagatctggcaacagatcttttttt

SEQ ID NO: 30 - RNA, 20 nt, synthetic construct
  misc_feature 1..20: note = tracrRNA Portion 2 sequence B
  source 1..20: mol_type = other RNA
aaagatgcaaatcttttttt

SEQ ID NO: 31 - RNA, 17 nt, synthetic construct
  misc_feature 1..17: note = tracrRNA Portion 2 - partial sequence A
  source 1..17: mol_type = other RNA
aaagatctggcaacaga

SEQ ID NO: 32 - RNA, 13 nt, synthetic construct
  misc_feature 1..13: note = tracrRNA Portion 2 - partial sequence B
  source 1..13: mol_type = other RNA
aaagatgcaaatc

SEQ ID NO: 33 - RNA, 22 nt, synthetic construct
  misc_feature 1..22: note = TRAC s91 spacer
  source 1..22: mol_type = other RNA
gctgtggcctggagcaacaaat

SEQ ID NO: 34 - RNA, 22 nt, synthetic construct
  misc_feature 1..22: note = PDCD1 s40 spacer
  source 1..22: mol_type = other RNA
aacacatcggagagcttcgtgc

SEQ ID NO: 35 - RNA, 22 nt, synthetic construct
  misc_feature 1..22: note = B2M s12 spacer
  source 1..22: mol_type = other RNA
gtatgcctgccgtgtgaaccat

SEQ ID NO: 36 - RNA, 22 nt, synthetic construct
  misc_feature 1..22: note = TRAC s35 spacer
  source 1..22: mol_type = other RNA
gaccctgccgtgtaccagctga

SEQ ID NO: 37 - RNA, 107 nt, synthetic construct
  misc_feature 1..107: note = OMNI-103 v2.2 TRAC S35 sgRNA
  source 1..107: mol_type = other RNA
gaccctgccgtgtaccagctgagtttgagagtagtgtaagaaattacactacaagttcaaataaaaatttattcaaatccatttgctacattgtgtagaattttttt

SEQ ID NO: 38 - RNA, 101 nt, synthetic construct
  misc_feature 1..101: note = OMNI-103 v2.3 TRAC S35 sgRNA
  source 1..101: mol_type = other RNA
gaccctgccgtgtaccagctgagtttgagagtagtggaaacactacaagttcaaataaaaatttattcaaatccatttgctacattgtgtagaattttttt

SEQ ID NO: 39 - RNA, 107 nt, synthetic construct
  misc_feature 1..107: note = OMNI-103 v2.2 B2M S12 sgRNA
  source 1..107: mol_type = other RNA
gtatgcctgccgtgtgaaccatgtttgagagtagtgtaagaaattacactacaagttcaaataaaaatttattcaaatccatttgctacattgtgtagaattttttt

SEQ ID NO: 40 - RNA, 101 nt, synthetic construct
  misc_feature 1..101: note = OMNI-103 v2.3 B2M S12 sgRNA
  source 1..101: mol_type = other RNA
gtatgcctgccgtgtgaaccatgtttgagagtagtggaaacactacaagttcaaataaaaatttattcaaatccatttgctacattgtgtagaattttttt